(19) United States
(12) Patent Application Publication
(10) Pub. No.: US 2023/0390279 A1
(43) Pub. Date: Dec. 7, 2023

(54) COMPOSITION AND METHOD FOR TREATING CANCER

(71) Applicant: NOVMETAPHARMA CO., LTD., Seoul (KR)

(72) Inventors: Keun Gyu Park, Daegu (KR); In Kyu Lee, Daegu (KR); Sung Jin Cho, Daegu (KR); Yeon Kyung Choi, Daegu (KR); Mi Jin Kim, Daegu (KR); Jung Wook Chin, Daegu (KR); Yong Hyun Jeon, Daegu (KR); Jin A Kim, Daegu (KR); Dong Su Kim, Daegu (KR); Hoe Yune Jung, Pohang-si (KR); Do Hyun Lee, Pohang-si (KR)

(73) Assignee: NOVMETAPHARMA CO., LTD., Seoul (KR)

(21) Appl. No.: 18/204,520

(22) Filed: Jun. 1, 2023

Related U.S. Application Data
(60) Provisional application No. 63/348,185, filed on Jun. 2, 2022.

(51) Int. Cl. A61K 31/495; A61K 39/395; A61P 35/00; A61K 31/7105; A61K 31/451; A61K 31/5377; A61K 31/47; A61K 31/506; A61K 31/519

(52) U.S. Cl. CPC A61K 31/495; A61K 39/3955; A61P 35/00; A61K 31/7105; A61K 31/519; A61K 31/5377; A61K 31/47; A61K 31/506; A61K 31/451

(57) ABSTRACT

A new use of estrogen-related receptor γ (ERRγ) inhibitor in enhancing cancer treatment and a pharmaceutical composition for inhibiting the resistance of cancer to tyrosine kinase inhibitors and enhancing an anticancer effect are disclosed. The pharmaceutical composition contains an ERRγ inhibitor as an active ingredient. The pharmaceutical composition for treating tyrosine kinase inhibitor-resistant advanced cancer. The composition can be administered in combination with tyrosine kinase inhibitor. A method for determining if a cancer is tyrosine kinase-resistant is also disclosed.

### CROSS REFERENCE TO RELATED APPLICATIONS

This application claims priority to U.S. Provisional Application No. 63/348,185 filed Jun. 2, 2022, of which the entire content is incorporated by reference herein.

### TECHNICAL FIELD

The disclosure relates to a use of an estrogen-related receptor γ (ERRγ) inhibitor or a composition comprising the same, as an active ingredient, in enhancing cancer treatments. The disclosure also relates to a method for cancer treatment or improving cancer treatments.

The disclosure also relates to a method for and a composition for use in treating tyrosine-kinas inhibitor resistant proliferative diseases.

### BACKGROUND ART

According to the World Health Organization (WHO), cancer is a leading cause of death worldwide, accounting for nearly 10 million deaths in 2020. The most common in 2020 (in terms of new cases of cancer) were breast, lung, colon and rectum, prostate, skin, and stomach, and the most common causes of cancer death in 2020 were lung, colon and rectum, liver, stomach, and breast.

A traditional approach to fight cancer is chemotherapy using cytotoxic drugs.

However, these cytotoxic agents are often poorly selective for cancer cells, subsequently leading to damage to normal cells that divide rapidly. Other treatment options include radiation therapy, targeted therapy, immunotherapy, hormonal therapy, and surgery. The choice of therapy depends upon various factors including the cancer type, severity of the tumor, the stage of the disease and general state of the patients.

Targeted therapeutic agents are generally prepared to show efficacy in patients by targeting malfunctioning proteins that cancer cells characteristically have. One example is tyrosine kinase inhibitors (TKIs). Tyrosine kinase inhibitors (TKIs) are target-specific inhibitors of abnormal protein tyrosine kinases (PTKs). TKIs have significant advantages over traditional chemotherapeutic agents, including high efficiency, low toxicity, and high specificity. Presently, TKIs are widely used for treating leukemia, non-small-cell lung cancer (NSCLC), renal cell carcinoma (RCC), gastrointestinal stromal tumor (GIST), breast cancer, and hepatocellular carcinoma (HCC), and their clinical application is rapidly growing. Uses of tyrosine kinase inhibitors (TKIs) has been responsible for effective therapeutic responses in patients. Currently, drug resistance is the main reason for limiting TKIs efficacy of cancer.

Therefore, there is a need for therapeutic methods and pharmaceutical compositions that can treat malignant tumors in patients resistant to TKI chemotherapy and/or enhancing cancer treatments such as TKI treatments and/or deter developing TKI resistance.

### SUMMARY

An aspect of the present disclosure provides a pharmaceutical composition for preventing or enhancing the treatment of tyrosine kinase inhibitor (TKI)-resistant cancer, which includes an ERRγ inhibitor as an active ingredient.

An aspect of the present disclosure provides a pharmaceutical composition for inhibiting TKI resistance in cancer, which includes an ERRγ inhibitor as an active ingredient.

Another aspect of the present disclosure provides a pharmaceutical composition for preventing or treating cancer, which includes an ERRγ inhibitor as an active ingredient, wherein the pharmaceutical composition is administered in combination with a TKI.

Another aspect of the present disclosure provides a method of screening a material that enhances the treatment of TKI-resistant cancer, which includes treating TKI-resistant cancer cells with a candidate material; and evaluating the activity or expression of ERRγ in the cells.

Yet another aspect of the present disclosure provides a kit for diagnosing TKI-resistant cancer, which includes an agent for measuring a level of mRNA of an estrogen-related receptor γ (ERRγ) gene or a protein expressed therefrom.

Yet another aspect of the present disclosure provides a method of providing information for diagnosis of TKI-resistant cancer, including the following steps:

(a) measuring an expression level of mRNA of an ERRγ gene or a protein expressed therefrom in a biological sample isolated from a first cancer patient to check whether cancer exhibits resistance to RAF inhibitor;

(b) measuring an expression level of mRNA of an ERRγ gene or a protein expressed therefrom in a biological sample isolated from a second patient with a cancer that is not resistant to a TKI or a subject without a cancer; and

(c) determining the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

Still another aspect of the present disclosure provides a method of providing information required to determine a therapeutic method for a first cancer patient, which includes the following steps:

(a) measuring an expression level of mRNA of an ERRγ gene or a protein expressed therefrom in a biological sample isolated from the first cancer patient;

(b) measuring an expression level of mRNA of an ERRγ gene or a protein expressed therefrom in a biological sample isolated from a second patient with a cancer that is not resistant to an RAF inhibitor or a subject without a cancer; and

(c) determining the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

Yet another aspect of the present disclosure provides a method of diagnosing or treating TKI-resistant cancer, which includes the following steps:

(a) measuring an expression level of mRNA of an ERRγ gene or a protein expressed therefrom in a biological sample isolated from a first cancer patient;

(b) measuring an expression level of mRNA of an ERRγ gene or a protein expressed therefrom in a biological sample isolated from a second patient with a cancer that is not TKI-resistant to or a subject without a cancer; and

(c) determining the first cancer patient in step (a) to be a patient with TKI-resistant cancer and applying another cancer therapeutic agent other than a TKI when the expression level of the mRNA of the ERRγ gene or the protein expressed therefrom measured in step (a) is higher than the expression level of the mRNA of the ERRγ gene or the protein expressed therefrom measured in step (b).

Yet another aspect of the present disclosure provides a method of preventing or enhancing the treatment of TKI-resistant cancer, which includes administering an ERRγ inhibitor to the patient with TKI-resistant cancer.

Yet another aspect of the present disclosure provides a method of inhibiting TKI resistance in cancer, which includes administering an ERRγ inhibitor to the patient with TKI-resistant cancer.

Still another aspect of the present disclosure provides a method of preventing or treating cancer, which includes administering an ERRγ inhibitor and a TKI to a cancer patient. In an embodiment, the cancer patient (or the first patient with cancer) may be previously treated with a TKI.

Yet another aspect of the present disclosure provides a combination comprising a first composition comprising an ERRγ inhibitor and a second composition comprising a TKI, said combination is for treating a cancer. In an embodiment, the cancer may be TKI-resistant.

Yet another aspect of the present disclosure provides a method for treating cancer in a subject, comprising administering an effective amount of a composition comprising an ERRγ inhibitor to the subject, wherein the subject has undergone, is undergoing, or is to receive a TKI treatment. The method may further comprise administering to the subject, sequentially or simultaneously, a TKI.

In the above pharmaceutical compositions, kits, screening methods, treatment methods, and diagnosing methods, TKIs include, but are not limited to, inhibitors of VEGFR (vascular endothelial growth factor receptor), FGFR (fibrblast growth factor receptor), PEGFR (platelet-derived growth factor receptor), EGFR (epidermal growth factor receptor), ABL (Abelson kinase), TIE (tyrosine kinase with immunoglobulin-like and EGF-like domain), Src kinase, JAK (Janus kinase), and the like. The TKIs may be selective or act on multiple targets. The TKIs may be a combination of different inhibitors to a same or different tyrosine kinase targets.

In the above pharmaceutical compositions, kits, screening methods, treatment methods, and diagnosing methods, the cancer may be breast cancer, prostate cancer, melanoma, colorectal cancers, glioma (including recurrent glioblastoma), lung cancer, liver cancer, kidney cancer, ovarian cancer, sarcoma, desmoid tumor (aggressive fibromatosis), or thyroid cancer.

In the above pharmaceutical compositions, kits, screening methods, treatment methods, and diagnosing methods, the TKI resistance may be innate resistance or acquired resistance. Therefore, in some embodiments, the cancer patient may be previously treated with a TKI inhibitor and developed resistance to the TKI inhibitor.

### BRIEF DESCRIPTION OF DRAWINGS

FIGS. 1A to 1C are diagrams showing the results of constructing a liver cancer cell line exhibiting resistance to sorafenib (Huh7: liver cancer cell line; Huh7-R: sorafenib-resistant liver cancer cell line; SK-Hep: liver cancer cell line; SK-Hep-R: sorafenib-resistant liver cancer cell line).

FIGS. 2A and 2B are diagrams showing that orphan nuclear receptor ERRγ increases in the sorafenib-resistant liver cancer cell lines Huh7-R (FIG. 2A) and SK-Hep-R FIG. 2B).

FIGS. 3A and 3B are diagrams showing the results of treating the sorafenib-resistant liver cancer cell lines Huh7-R (FIG. 3A) and SK-Hep-R (FIG. 3B) with a compound (DN200434) of Formula 1 that is an inverse agonist against ERRγ, indicating that reactive oxygen species (ROS) increased in each of the sorafenib-resistant liver cancer cell lines.

FIGS. 4A and 4B are diagrams showing that the sorafenib-resistant liver cancer cell lines Huh7-R (FIG. 4A) and SK-Hep-R (FIG. 4B) which did not have an anticancer effect when the cell lines are treated with sorafenib alone have reduced cell proliferation when the cell lines are treated with the compound of Formula 1 (DN200434) and sorafenib at the same time.

FIG. 5A is an image showing that the size of a tumor remarkably decreases in an animal model (xenograft) derived from the sorafenib-resistant liver cancer cell line Huh7-R when the compound of Formula 1 (DN200434) is administered in combination with sorafenib, compared to the control (a sorafenib-alone-administered group).

FIG. 5B is a graph showing that the weight of a tumor remarkably decreases in the animal model derived from the sorafenib-resistant liver cancer cell line Huh7-R when the compound of Formula 1 (DN200434) is administered in combination with sorafenib, compared to the control (a sorafenib-alone-administered group).

FIG. 5C is a diagram showing that the volume of a tumor remarkably decreases in the animal model derived from the sorafenib-resistant liver cancer cell line Huh7-R when the compound of Formula 1 (DN200434) is administered in combination with sorafenib, compared to the control (Huh7-R-Con; a sorafenib-alone-administered group).

FIG. 5D is a diagram showing that there is no change in body weight between groups as a result of an experiment using the animal model derived from the sorafenib-resistant liver cancer cell line Huh7-R.

FIG. 6A is an image showing that the size of a tumor remarkably decreases in an animal model (xenograft) derived from the sorafenib-resistant liver cancer cell line SK-Hep-R when the compound of Formula 1 (DN200434) is administered in combination with sorafenib, compared to the control (a sorafenib-alone-administered group).

FIG. 6B is a graph showing that the weight of a tumor remarkably decreases in the animal model derived from the sorafenib-resistant liver cancer cell line SK-Hep-R when the compound of Formula 1 (DN200434) is administered in combination with sorafenib, compared to the control (a sorafenib-alone-administered group).

FIG. 6C is a diagram showing that the volume of a tumor remarkably decreases in the animal model derived from the sorafenib-resistant liver cancer cell line SK-Hep-R when the compound of Formula 1 (DN200434) is administered in combination with sorafenib, compared to the control (Huh7-R-Con; a sorafenib-alone-administered group).

FIG. 6D is a diagram showing that there is no change in body weight between groups as a result of an experiment using the animal model derived from the sorafenib-resistant liver cancer cell line SK-Hep-R.

FIG. 7 shows structures of exemplary non-limiting TKIs.

FIG. 8 shows unexpectedly superior synergic anticancer effects by a combination of DN434 and bosutinib in in vitro experiments employing A549 cells.

FIG. 9 shows unexpectedly superior synergic anticancer effects by a combination of DN434 and gefitinib in in vitro experiments employing HCC827 cells and PC9 cells.

FIG. 10 shows unexpectedly superior synergic anticancer effects by a combination of DN434 and lenvatinib in in vitro experiments employing Huh-7 cells and SK-Hep-1 cells.

FIG. 11 shows unexpectedly superior synergic anticancer effects by a combination of DN434 and ceritinib in in vitro experiments employing Panc1 cells.

FIG. 12 shows unexpectedly superior synergic anticancer effects by a combination of DN434 and ruxolitinib in in vitro experiments employing SKOV-3 cells.

FIG. 13 shows cancer treatment effects of DN434 in erlotinib-resistant cancer in in vitro experiments employing erlotinib-resistant HCC837 cells.

### DETAILED DESCRIPTION

### Definitions

The general terms used herein are defined with the following meanings, unless explicitly stated otherwise:

The terms “a” and “an” and “the” and similar references in the context of describing the disclosure (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “about” or “approximately” shall have the meaning of within 10%, more preferably within 5%, of a given value or range.

The term “IC₅₀” refers to an amount, concentration or dosage of a particular test compound that achieves a 50% inhibition of a maximal response, such as cell growth or proliferation, measured via any of the in vitro or cell based assay described herein.

The terms “comprising” and “including” are used herein in their open-ended and non-limiting sense unless otherwise noted. The terms “consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “hydrate” means a compound provided herein or a salt thereof, that further includes a stoichiometric or non-stoichiometric amount of water bound by non-covalent intermolecular forces.

The term “solvate” means a solvate formed from the association of one or more solvent molecules to a compound provided herein. The term “solvate” includes hydrates (e.g., mono-hydrate, dihydrate, trihydrate, tetrahydrate and the like).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “administration” refers to introducing an agent of the present disclosure into a subject or patient. One preferred route of administration of the agents is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

The terms “co-administration” or “combined administration” or “combined use” or “in combination with” are meant to encompass administration of two therapeutic agents (for example, the ERRγ inhibitor provided herein and another anti-cancer agent) to a single subject in need thereof (e.g. a patient), and are intended to include treatment regimens in which the two agents are administered simultaneously, concurrently, or sequentially with no specific time limits. In embodiments, both agents are present in the cell or in the patient's body at the same time or exert their biological or therapeutic effect at the same time. In embodiments, the two therapeutic agents are in the same composition or unit dosage form. In another embodiments, the two therapeutic agents are in separate compositions or unit dosage forms.

The term “jointly therapeutically active” or “joint therapeutic effect” as used herein means that the therapeutic agents may be given separately (in a chronologically staggered manner, especially a sequence-specific manner) in such time intervals that they prefer, in a mammal, a warm-blooded animal, especially human, to be treated, still show a (preferably synergistic) interaction (joint therapeutic effect). Whether this is the case can, inter alia, be determined by following the blood levels, showing that both compounds are present in the blood of the human to be treated at least during certain time intervals.

The term “synergistic effect” as used herein refers to action of two therapeutic agents such as, for example, the ERRγ inhibitors provided herein and bosutinib as a TKI, producing an effect, for example, slowing the symptomatic progression of a proliferative disease, particularly cancer, or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The term “anti-cancer agents” refers to anti-metabolites (e.g., 5-fluoro-uracil, methotrexate, fludarabine), antimicrotubule agents (e.g., vinca alkaloids such as vincristine, vinblastine; taxanes such as paclitaxel, docetaxel), alkylating agents (e.g., cyclophosphamide, melphalan, carmustine, nitrosoureas such as bischloroethylnitrosurea and hydroxyurea), platinum agents (e.g. cisplatin, carboplatin, oxaliplatin, JM-216 or satraplatin, CI-973), anthracyclines (e.g., doxorubicin, daunorubicin), antitumor antibiotics (e.g., mitomycin, idarubicin, adriamycin, daunomycin), topoisomerase inhibitors (e.g., etoposide, camptothecins), anti-angiogenesis agents (e.g. Sutent® and Bevacizumab) or any other cytotoxic agents, (estramustine phosphate, prednimustine), hormones or hormone agonists, antagonists, partial agonists or partial antagonists, kinase inhibitors, radiation treatment, or therapeutic antibody.

The term “pharmaceutical composition” or “formulation” is defined herein to refer to a mixture or solution containing at least one therapeutic agent to be administered to a subject, e.g., a mammal or human, in order to prevent or treat a particular disease or condition affecting the subject or patient including mammal or human.

The term “pharmaceutically acceptable excipient” or “pharmaceutically acceptable carrier” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use.

The term “treating” or “treatment” as used herein comprises a treatment relieving, reducing or alleviating at least one symptom in a subject or effecting a delay of progression of a disease. For example, treatment can be the diminishment of one or several symptoms of a disorder or complete eradication of a disorder, such as cancer. Within the meaning of the present disclosure, the term “treat” also denotes to arrest, delay the onset (i.e., the period prior to clinical manifestation of a disease) and/or reduce the risk of developing or worsening a disease, and/or suppressing the increase in tumor volume or size, or reducing the tumor volume or size.

The term “enhancing the treatment of” as used herein refers to all types of actions for improving or beneficially changing the symptoms of TKI-resistant cancer by administration of the pharmaceutical composition of the present disclosure after cancer has acquired resistance to TKI.

The terms “ameliorate,” “ameliorating” and grammatical variations thereof mean to decrease the severity of the symptoms of a disease in a subject.

The term “protect” is used herein to mean prevent delay or treat, or all, as appropriate, development or continuance or aggravation of a disease in a subject, e.g., a mammal or human.

The term “prevent”, “preventing” or “prevention” as used herein comprises the prevention of at least one symptom associated with or caused by the state, disease or disorder being prevented.

The term “effective amount” or “therapeutically effective amount” or “pharmaceutically effective amount” of an active pharmaceutical compound (APC) or pharmaceutical compositions containing APC is an amount of Compound A or composition that is sufficient to effect beneficial or desired results when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an agent or composition will be that amount of the agent or composition, which is the lowest dose effective to produce the desired effect. The effective dose of an agent or composition may be administered as two, three, four, five, six or more sub-doses, administered separately at appropriate intervals throughout the day.

The term “subject” or “patient” as used herein includes animals, which are capable of suffering from or afflicted with a cancer or any disorder involving, directly or indirectly, a cancer. Examples of subjects include mammals, e.g., humans, dogs, cows, horses, pigs, sheep, goats, cats, mice, rabbits, rats and transgenic non-human animals. In the preferred embodiment, the subject is a human, e.g., a human suffering from, at risk of suffering from, or potentially capable of suffering from cancers.

The term “inverse agonist” or “antagonist” as used herein refers to a molecule capable of directly or indirectly reducing the biological activity of a receptor, and includes molecules capable of reducing the action of a ligand when used in conjunction with the ligand of the receptor, but the present disclosure is not limited thereto.

The terms “miRNA,” “siRNA,” and “shRNA” as used herein refer to nucleic acid molecules that mainly bind to mRNA transcribed from a target gene to mediate RNA interference or gene silencing, thereby inhibiting the translation of mRNA. Because the miRNA, siRNA, and shRNA may inhibit the expression of the target gene at a translational level, they can be used in an efficient gene knockdown method or gene therapy method.

The term “antisense oligonucleotide” as used herein refers to a DNA or RNA or a derivative thereof containing a nucleic acid sequence complementary to a specific mRNA sequence. In this case, the antisense oligonucleotide may bind to a complementary sequence in mRNA, thereby inhibiting the translation of mRNA into a protein.

The term “antibody” as used herein refers to a proteinaceous molecule capable of specifically binding to an antigenic site of a protein or peptide molecule. In this case, a protein encoded by the marker gene may be obtained by cloning each gene into an expression vector according to a conventional method, and such an antibody may be prepared from the obtained protein using a conventional method.

The term “aptamer” as used herein refers to a nucleic acid molecule that has binding activity for a predetermined target molecule. The aptamer may be RNA, DNA, a modified nucleic acid, or a mixture thereof, and may be in a linear or cyclic form. In general, chemical synthesis and mass production may be easier as the nucleotide sequence constituting the aptamer gets shorter and it is known that the aptamer has an excellent advantage in terms of costs, is easily chemically modified, and exhibits excellent in vivo stability and low toxicity.

### ERRγ Inhibitors and Agent for Inhibiting ERRγ Gene Expression

In the pharmaceutical compositions, combinations, kits, screening methods, treatment methods, and diagnosing methods, ERRγ inverse agonist (or an inhibitor or antagonist) may be a compound of the following formula A:

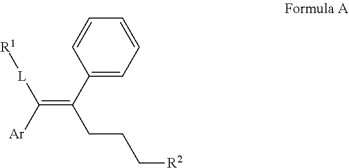

wherein

L is (C6-C20)arylene, (C3-C20)heteroarylene, or (C3-C20)fused heterocycle;

R¹ is (C3-C20)heterocycloalkyl, (C3-C20)heteroaryl, —O—(CH₂)ₘ—R¹¹, —(CH₂)ₘ—R¹², —NH—(CH₂)ₘ—R¹³, —NHCO—(CH₂)ₙ—R¹⁴, or —SiR¹⁶R¹⁷—(CH₂)ₘ—R¹⁵;

R¹¹ to R¹⁵ are independently of one another (C3-C20)heterocycloalkyl;

R¹⁶ and R¹⁷ are independently of each other (C1-C20)alkyl;

m is an integer of 1 to 3; and

n is an integer of 0 or 1;

Ar is (C6-C20)aryl or (C3-C20)heteroaryl, in which the aryl or heteroaryl of Ar may be further substituted by one or more selected from the group consisting of hydroxy, halogen, (C1-C20)alkyl, halo (C1-C20)alkyl, (C1-C20)alkoxy, nitro, cyano, —NR²¹R²², (C1-C20)alkylcarbonyloxy, (C1-C20)alkylcarbonylamino, guanidino, —SO₂—R²³, and —OSO₂—R²⁴;

R²¹ and R²² are independently of each other hydrogen, (C1-C20)alkylsulfonyl, or (C3-C20)cycloalkylsulfonyl;

R²³ and R²⁴ are independently of each other (C1-C20)alkyl, halo(C1-C20)alkyl, or (C3-C20)cycloalkyl;

R² is hydroxy, halogen, (C1-C20)alkylcarbonyloxy, or (C1-C20)alkylsulfonyloxy;

the heterocycloalkyl or heteroaryl of R¹ and the heterocycloalkyl of R¹¹ to R¹⁵ may be further substituted by one or more selected from the group consisting of (C1-C20)alkyl, (C3-C20)cycloalkyl, (C2-C20)alkenyl, amidino, (C1-C20)alkoxycarbonyl, hydroxy, hydroxy(C1-C20)alkyl, and di(C1-C20)alkylamino(C1-C20)alkyl; and

the heterocycloalkyl and heteroaryl contains one or more heteroatoms selected from the group consisting of N, O and S, and the heterocycloalkyl is a saturated or unsaturated mono-, bi-, or spirocycle having a carbon atom or nitrogen atom in a ring as a binding site, an isomer, or a pharmaceutically acceptable salt thereof, or a solvate including a hydrate.

The compound of Formula A and their preparation methods are disclosed in U.S. application Ser. No. 16/677,596 or U.S. Pat. No. 10,934,303, of which the contents are incorporated by reference in their entireties.

According to one embodiment of the present disclosure, the inverse agonist against ERRγ may be a compound of the following Formulas 1a-1d, a pharmaceutically acceptable salt thereof, or an isomer, or a solvate (including a hydrate) thereof, but is not limited to the compound as long as it is an inverse agonist against ERRγ that exhibits an effect equivalent to the compound:

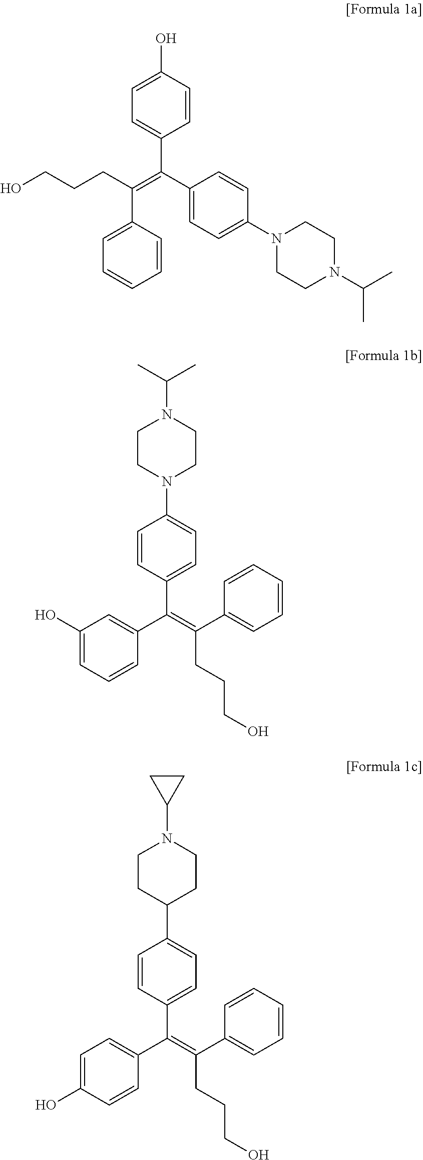 [SMILES: CC(C)N1CCN(c2ccc(/C(=C(/CCCO)c3ccccc3)c3ccc(O)cc3)cc2)CC1 ; CC(C)N1CCN(c2ccc(/C(=C(/CCCO)c3ccccc3)c3cccc(O)c3)cc2)CC1 ; OCCC/C(=C(\c1ccc(O)cc1)c1ccc(C2CCN(C3CC3)CC2)cc1)c1ccccc1]

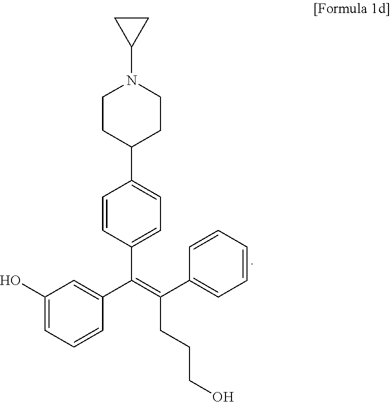 [SMILES: OCCC/C(=C(/c1ccc(C2CCN(C3CC3)CC2)cc1)c1cccc(O)c1)c1ccccc1]

In certain embodiments, the ERRγ inverse agonist may be (E)-5-(4-hydroxyphenyl)-5-(4-(4-isopropylpiperazin-1-yl)phenyl)-4-phenylpent-4-en-1-ol of Formula 1a (DN434 or DN200434), or a pharmaceutically acceptable salt thereof or an isomer thereof, or a solvate thereof.

The present inventors surprisingly found that the particular estrogen-related receptor γ (ERRγ) inhibitors described herein have effective anticancer effects in TKI-resistant cancers (e.g., Example 4 and FIG. 13). And combinations of the ERRγ inhibitors and TKIs exhibited remarkable synergistic anticancer effects in in vitro experiments, compared to

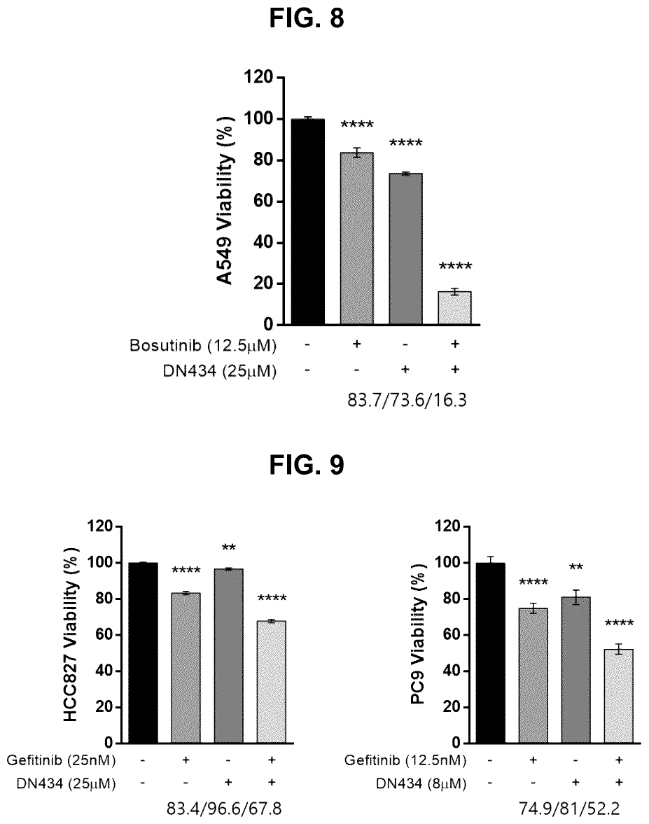

ERRγ inhibitor alone and TKI alone (e.g., Example 5 and FIGS. 8-12) Without bounding to a particular theory, the effective anticancer effects in TKI-resistant cancers and the remarkable synergistic effects may be attributed to the ERRγ inhibitor's action of enhancing the susceptibility to the anticancer drug TKI, inhibiting the proliferation of TKI-resistant cancer cells, and significantly reducing the size of cancer.

Therefore, the ERRγ inhibitors could be effectively used, on its own or in combination with TKIs, for treating advanced TKI-resistant cancer.

The compound of the present disclosure may be used in the form of a pharmaceutically acceptable salt. In this case, an acid addition salt formed by a pharmaceutically acceptable free acid may be used as the salt.

The acid addition salt may be obtained from inorganic acids such as hydrochloric acid, nitric acid, phosphoric acid, sulfuric acid, hydrobromic acid, hydroiodic acid, nitrous acid, or phosphorous acid, and aliphatic mono- and di-carboxylates, phenyl-substituted alkanoates, hydroxy alkanoates, and alkanedioates, and non-toxic organic acids such as aromatic acids, aliphatic and aromatic sulfonic acids, and the like.

Such a pharmaceutically non-toxic salt includes sulfate, pyrosulfate, bisulfate, sulfite, bisulfite, nitrate, phosphate, monohydrogen phosphate, dihydrogen phosphate, metaphosphate, pyrophosphate chloride, bromide, iodide, fluoride, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, β-hydroxybutyrate, glycolate, malate, tartrate, methanesulfonate, propanesulfonate, naphthalene-1-sulfonate, naphthalene-2-sulfonate, or mandelate.

The acid addition salt according to the present disclosure may be prepared by a conventional method, for example, by dissolving the compound of Formula A in an aqueous acid solution, and precipitating the salt in a water-miscible organic solvent such as methanol, ethanol, acetone, or acetonitrile. Also, the acid addition salt may be prepared by evaporating the solvent or excess acid from the mixture and drying the mixture, or by filtering the precipitated salt by suction.

Also, a pharmaceutically acceptable metal salt may be prepared using a base. An alkali metal or alkaline earth metal salt is obtained, for example, by dissolving the compound of Formula A in an excess alkali metal hydroxide or alkaline earth metal hydroxide solution, filtering the undissolved compound salt, and evaporating and drying the filtrate. In this case, it may be pharmaceutically suitable to prepare a sodium, potassium, or calcium salt as the metal salt. A silver salt corresponding to the metal salt is obtained by reacting an alkali metal or alkaline earth metal salt with a suitable silver salt (e.g., silver nitrate).

All isomers, hydrates and solvates that can be prepared by conventional methods, as well as pharmaceutically acceptable salts thereof, may be included in the scope of the compound of the present disclosure.

In embodiments, the agent for inhibiting ERRγ protein activity may include, an inverse agonist or an antagonist against ERRγ, or an antibody or an aptamer capable of specifically binding to ERRγ, but the present disclosure is not limited thereto. According to another embodiment of the present disclosure, the agent for inhibiting the expression of the ERRγ gene may be selected from the group consisting of miRNA, siRNA, shRNA, and an antisense oligonucleotide, all of which specifically bind to mRNA of the gene, but the present disclosure is not limited thereto.

ERRγ gene may comprise or consist of a nucleotide sequence encoding the ERRγ, as reported as NP_001127757.1 (nucleotide sequence NM_001134285.2 [P62508-2]), NP_001230434.1 ((nucleotide sequence NM_001243505.1), NP_001230435.1 (nucleotide sequence NM_001243506.1), NP_001230436.1 (nucleotide sequence NM_001243507.1 [P62508-4]), NP_001230438.1 (nucleotide sequence NM_001243509.1 [P62508-2]), NP_001230439.1 (nucleotide sequence NM_001243510.2 [P62508-2]), NP_001230440.1 (nucleotide sequence NM_001243511.2 [P62508-2]), NP_001230441.1 (nucleotide sequence NM_001243512.1 [P62508-2]), NP_001230442.1 (nucleotide sequence NM_001243513.1 [P62508-2]), NP_001230443.1 (nucleotide sequence NM_001243514.1 [P62508-2]), NP_001230444.1 (nucleotide sequence NM_001243515.1 [P62508-2]), NP_001230447.1 (nucleotide sequence NM_001243518.1 [P62508-5]), NP_001230448.1 (nucleotide sequence NM_001243519.1 [P62508-2]), NP_001429.2 (nucleotide sequence NM_001438.3 [P62508-1]), NP_996317.1 (nucleotide sequence NM_206594.2 [P62508-2]), NP_996318.1,(nucleotide sequence NM_206595.2 [P62508-2]), XP_011507569.1 (nucleotide sequence XM_011509267.1 [P62508-5]), XP_011507570.1,(nucleotide sequence XM_011509268.2 [P62508-5]), XP_011507571.1 (nucleotide sequence XM_011509269.2 [P62508-5]), XP_011507576.1 (nucleotide sequence XM_011509274.1 [P62508-3]), XP_011507577.1 (nucleotide sequence XM_011509275.1 [P62508-3]), XP_011507578.1 (nucleotide sequence XM_011509276.1 [P62508-3]), XP_011507579.1 (nucleotide sequence XM_011509277.1 [P62508-3]), XP_011507580.1 (nucleotide sequence XM_011509278.1 [P62508-3]), XP_011507581.1 (nucleotide sequence XM_011509279.1 [P62508-3]), XP_011507582.1 (nucleotide sequence XM_011509280.2 [P62508-3]), XP_016856120.1 (nucleotide sequence XM_017000631.1 [P62508-3]), XP_016856121.1 (nucleotide sequence XM_017000632.1 [P62508-3]), XP_016856122.1 (nucleotide sequence XM_017000633.1 [P62508-3]), XP_016856123.1 (nucleotide sequence XM_017000634.1 [P62508-3]), XP_016856124.1 (nucleotide sequence XM_017000635.1 [P62508-3]), XP_016856125.1 (nucleotide sequence XM_017000636.1 [P62508-3]), XP_016856126.1 (nucleotide sequence XM_017000637.1 [P62508-3]), XP_016856127.1 (nucleotide sequence XM_017000638.1 [P62508-2]), XP_016856128.1 (nucleotide sequence XM_017000639.1 [P62508-2]), XP_016856129.1 (nucleotide sequence XM_017000640.1), XP_016856130.1 (nucleotide sequence XM_017000641.1 [P62508-2]), XP_016856131.1 (nucleotide sequence XM_017000642.1), XP_016856132.1 (nucleotide sequence XM_017000643.1), XP_016856133.1 (nucleotide sequence XM_017000644.1 [P62508-2]), XP_016856134.1 (nucleotide sequence XM_017000645.1 [P62508-2]), XP_016856135.1 (nucleotide sequence XM_017000646.1), XP_016856136.1 (nucleotide sequence XM_017000647.1), XP_016856137.1 (nucleotide sequence XM_017000648.1), XP_016856138.1 (nucleotide sequence XM_017000649.1), or its natural variant form VAR_019229 (T50M), but are not limited thereto.

The agent capable of inhibiting ERRγ protein activity or ERRγ gene expression may be used to inhibit the resistance to a TKI in the prevention or treatment of cancer.

According to one embodiment of the present disclosure, the composition may enhance the drug susceptibility of cancer to a TKI or may enhance an anticancer effect of a TKI on cancer, but the present disclosure is not limited thereto.

Therefore, according to still another embodiment of the present disclosure, the composition comprising an ERRγ inhibitor or an agent for inhibiting the expression of the ERRγ gene may be administered simultaneously with a TKI, separately, or sequentially, but the present disclosure is not limited thereto. And, the method for treating cancer or preventing TKI resistance in cancer, comprise administering the composition comprising an ERRγ inhibitor or an agent for inhibiting the expression of the ERRγ gene and further comprise administering a TKI, simultaneously, separately, or sequentially.

The nature of proliferative diseases is multifactorial, and drugs with different mechanisms of action may be combined. For example, the pharmaceutical composition may be administered alone or in combination with known chemotherapy, radiation therapy, cancer immunotherapy, or symptomatic treatment. In embodiments, the chemotherapy may include, but is not limited to, bosutinib (src tyrosine kinase inhibitor), gefitinib (EGFR tyrosine kinase inhibitor), lenvatinib (Multiple kinase inhibitor targeting VEGFR1, 2, 3, FGFR1, 2, 3, and others), ceritinib (ALK inhibitor), ruxolitinib (JAK inhibitor), erlotinib, sunitinib, pazopanib, vandetanib, axitinib, cediranib, vatalanib, motesanib, RAF265, sorafenib, afatinib, osimertinib, lazertinib, SB590885, PLX8394, PLX 4720, PLX4032, GDC-0879, ZM 336372, dabrafenib, encorafenib, CCT3833/BAL3833, LY3009120, BGB-283 (lifirafenib), belvarafenib, TAK-580, trametinib, RO5126766, trametiglue, nazartinib, mavelertinib, azertinib, naquotinib, olmutinib, rociletinib, or combinations thereof. The another treatment can be a therapeutic antibody, wherein the therapeutic antibody is cetuximab, panitumumab, nimotuzumab or necitumumab. The another treatment can be an immunotherapy comprising, but not limited to, a therapeutic antibody, wherein the therapeutic antibody is cetuximab, panitumumab, nimotuzumab, or necitumumab.

### Tyrosine Kinase Inhibitors and Resistance

Tyrosine kinase inhibitors (TKIs) are target-specific inhibitors of abnormal protein tyrosine kinases (PTKs). TKIs have significant advantages over traditional chemotherapeutic agents, including high efficiency, low toxicity, and high specificity.

TKIs include, but are not limited to, inhibitors of VEGFR (vascular endothelial growth factor receptor), FGFR (fibroblast growth factor receptor), PEGFR (platelet-derived growth factor receptor), EGFR (epidermal growth factor receptor), ABL (Abelson kinase), TIE (tyrosine kinase with immunoglobulin-like and EGF-like domain), Src kinase, JAK (Janus kinase), and the like. The TKIs may be selective or act on multiple targets. The TKIs may be a combination of different inhibitors to a same or different tyrosine kinase targets.

Non-limiting exemplary TKI inhibitors include, but are not limited to, bosutinib (src tyrosine kinase inhibitor), gefitinib (EGFR tyrosine kinase inhibitor), lenvatinib (Multiple kinase inhibitor targeting VEGFR1, 2, 3, FGFR1, 2, 3, and others), ceritinib (ALK inhibitor), ruxolitinib (JAK inhibitor), erlotinib, sunitinib, pazopanib, vandetanib, axitinib, cediranib, vatalanib, motesanib, RAF265, sorafenib, afatinib, osimertinib, lazertinib, SB590885, PLX8394, PLX 4720, PLX4032, GDC-0879, ZM 336372, dabrafenib, encorafenib, CCT3833/BAL3833, LY3009120, BGB-283 (lifirafenib), belvarafenib, TAK-580, trametinib, RO5126766, trametiglue, nazartinib, mavelertinib, azertinib, naquotinib, olmutinib, rociletinib, or combinations thereof.

The TKI-resistance may be innate or acquired. Thus, in embodiments of the present disclosure, cancer may be characterized by resistance to at least one tyrosine kinase inhibitor (TKI) due to: (a) at least one mutation in a gene encoding a protein that is a target of at least one TKI; or (b) the presence of at least one additional gene in either a wild-type or mutated state encoding a product that confers resistance to the therapeutic effects of at least one TKI. Patients may have innate or acquired mutations or polymorphisms that affect the apoptotic response to TKI. In some embodiments, polymorphisms that affect the apoptotic response to TKI include, but are not necessarily limited to, polymorphisms in the gene BCL2L11 (also known as BM, which encodes a BH3-only protein that is a BCL-2 family member. In some embodiments, mutations may include EGFR L858R, EGFR L858R/T790M, EGFR L858R/C797S, L858R/T790M/C797S, EGFR Del19, EGFR Del19/T790M, EGFR Del19/C797S, or EGFR Del19/T790M/C797S.

According to an aspect, the cancer may be breast cancer, prostate cancer, melanoma, colorectal cancers, glioma (including recurrent glioblastoma), lung cancer, liver cancer, kidney cancer, ovarian cancer, sarcoma, pancreatic, desmoid tumor (aggressive fibromatosis), or thyroid cancer.

In another aspect, cancer may include pseudomyxoma, intrahepatic cholangiocarcinoma, hepatoblastoma, liver cancer, thyroid cancer, colon cancer, testis cancer, myelodysplastic syndrome, glioblastoma, oral cancer, lip cancer, mycosis fungoides, acute myeloid leukemia, acute lymphocytic leukemia, basal cell carcinoma, epithelial ovarian cancer, ovarian seminoma, male breast cancer, brain cancer, pituitary adenoma, multiple myeloma, gallbladder cancer, cholangiocarcinoma, colorectal cancer, chronic myeloid leukemia, chronic lymphocytic leukemia, retinoblastoma, choroidal melanoma, ampullar of vater cancer, bladder cancer, peritoneal cancer, parathyroid cancer, adrenal cancer, nasal and paranasal cavity cancer, non-small cell lung cancer, tongue cancer, astrocytoma, small cell lung cancer, pediatric brain cancer, pediatric lymphoma, pediatric leukemia, small intestine cancer, meningioma, esophageal cancer, glioma, renal pelvis cancer, renal cancer, heart cancer, duodenal cancer, malignant soft tissue cancer, malignant bone cancer, malignant lymphoma, malignant mesothelioma, malignant melanoma, eye cancer, vulvar cancer, ureteral cancer, urethral cancer, cancer of unknown primary site, gastric lymphoma, gastric cancer, gastric carcinoid, gastrointestinal stromal tumor, Wilms' tumor, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid, vaginal cancer, spinal cancer, vestibular schwannoma, pancreatic cancer, salivary gland cancer, Kaposi's sarcoma, Paget's disease, tonsillar cancer, squamous cell cancer, adenocarcinoma of lung, lung cancer, squamous cell lung cancer, skin cancer, anal cancer, rhabdomyosarcoma, laryngeal cancer, pleura cancer, and thymic cancer. The cancer may have mutations.

### Pharmaceutical Composition and Treatment Methods

An aspect of the present disclosure also pertains to a pharmaceutical combination comprising: (a) an ERRγ inhibitor, or a pharmaceutically acceptable salt thereof, and (b) one or more TKI, or a pharmaceutically acceptable salt thereof, and optionally at least one pharmaceutically acceptable carrier for use in the preparation of a pharmaceutical composition or medicament for the treatment or prevention of a proliferative disease in a subject in need thereof.

Another aspect of the present disclosure also pertains to a pharmaceutical combination comprising: (a) an ERRγ inhibitor of Formula 1a, 1b, 1c, or 1d, or a pharmaceutically acceptable salt thereof, and (b) a TKI selected from bosutinib, gefitinib, lenvatinib, ceritinib, ruxolitinib, erlotinib, sunitinib, pazopanib, vandetanib, axitinib, cediranib, vatalanib, motesanib, RAF265, sorafenib, afatinib, osimertinib, lazertinib, SB590885, PLX8394, PLX 4720, PLX4032, GDC-0879, ZM 336372, dabrafenib, encorafenib, CCT3833/BAL3833, LY3009120, BGB-283 (lifirafenib), belvarafenib, TAK-580, trametinib, RO5126766, trametiglue, nazartinib, mavelertinib, azertinib, naquotinib, olmutinib, rociletinib, or combinations thereof, and optionally at least one pharmaceutically acceptable carrier useful for treating or preventing a proliferative disease in a subject in need thereof.

An aspect of the present disclosure further provides a commercial package comprising, as therapeutic agents, a combination comprising: (a) an ERRγ inhibitor, or a pharmaceutically acceptable salt thereof, and (b) one or more TKI, or a pharmaceutically acceptable salt thereof, and optionally at least one pharmaceutically acceptable carrier for use in the preparation of a pharmaceutical composition, together with instructions for simultaneous, separate or sequential administration thereof for use in the delay of progression or treatment of a proliferative disease.

The above combinations are also provided for simultaneous, separate or sequential administration, in particular for treating or preventing a proliferative disease.

The combination of the two compounds according to the aspects, optionally comprising another chemotherapeutic agent, can be used for the treatment of proliferation disease or cancer. The nature of proliferative diseases is multifactorial, and drugs with different mechanisms of action may be combined. However, just considering any combination of therapeutic agents having different mode of action does not necessarily lead to combinations with advantageous effects. The administration of a pharmaceutical combination of the disclosure may result not only in a beneficial effect, e.g. a synergistic therapeutic effect, e.g. with regard to alleviating, delaying progression of or inhibiting the symptoms, but also in further surprising beneficial effects, e.g. fewer side-effects, an improved quality of life or a decreased morbidity, compared with a monotherapy applying only one of the pharmaceutically therapeutic agents used in the combination of the disclosure. A further benefit is that lower doses of the therapeutic agents of the combination of the disclosure can be used, for example, that the dosages may be smaller, that the drugs are applied less frequently, or can be used in order to diminish the incidence of side-effects observed with one of the combination partners alone.

The active ingredient compounds, i.e., the ERRγ inhibitor and the TKI according to the embodiments are preferably formulated or used to be jointly (prophylactically or especially therapeutically) active. And, for example, the compounds may be given separately or sequentially (in a chronically staggered manner, especially a sequence-specific manner) in such time intervals that they preferably, in the subject to be treated, and still show a (preferably synergistic) interaction (joint therapeutic effect). A joint therapeutic effect can, among others, be determined by following the blood levels, showing that both compounds are present in the blood of the human to be treated at least during certain time intervals, but this is not to exclude the case where the compounds are jointly active although they are not present in blood simultaneously.

Suitable pharmaceutical compositions or formulations include for example tablets, capsules, suppositories, solutions, particularly solutions for injection (i.v., s.c., i.m.) and infusion, syrups, elixirs, emulsions or dispersible powders. The content of the pharmaceutically active compounds may be in the range from 0.1 to 90 wt. %, preferably 0.5 to 50 wt. % of total weight or volume of the composition.

The pharmaceutical combination composition can be either administered in a single formulation or unit dosage form, administered concurrently, but optionally separately, or administered sequentially by any suitable route. The unit dosage form may also be a fixed combination

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly, the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules comprising one or more active substances or combinations of active substances may for example be prepared by mixing the active substances with inert carriers such as lactose or sorbitol and packing them into gelatine capsules.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The compositions are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavor enhancers or colorings in addition to the excipients mentioned above.

For parenteral use, solutions of the active ingredients with suitable liquid carriers may be used.

The dosage of the pharmaceutical composition is an amount effective for treating or preventing an individual, a subject, or a patient, and may be orally or parenterally administered as desired. Upon oral administration, 0.01 to 1000 per kg of body weight per day based on the active ingredient mg, more specifically 0.1 to 1000 mg, to be administered in an amount of 0.01 to 100 mg per kg body weight per day, more specifically 0.1 to 50 mg based on the active ingredient during parenteral administration. It may be administered in divided doses of 1 to several times. Dosages for a particular individual, subject, or patient should be determined in light of several relevant factors such as the patient's weight, age, sex, health condition, diet, time of administration, mode of administration, severity of the disease and can be appropriately added or subtracted by a specialist. It is to be understood that such doses are not intended to limit the scope of the disclosure in any aspect. A physician or veterinarian having ordinary skill in the art can readily determine and prescribe the effective amount of the required pharmaceutical composition. For example, a physician or veterinarian may use a dose of a compound of the disclosure for use in a pharmaceutical composition, starting at a lower level than required to achieve the desired therapeutic effect, and gradually increasing the dosage until the desired effect is achieved.

### Diagnosing Methods and Kits

According to yet another aspect of the present disclosure, there is provided a kit for diagnosing TKI-resistant cancer, which includes an agent for measuring a level of mRNA of ERRγ gene or a protein expressed therefrom.

According to one embodiment of the present disclosure, the agent for measuring a level of mRNA of the gene may include a pair of primers, a probe, or an antisense nucleotide, which specifically binds to the gene, but the present disclosure is not limited thereto.

According to yet another embodiment of the present disclosure, the agent for measuring a level of the protein may include an antibody or an aptamer specific for the protein, but the present disclosure is not limited thereto.

According to yet another embodiment of the present disclosure, the kit may include an RT-PCR kit, a competitive RT-PCR kit, a real-time RT-PCR kit, a DNA chip kit, or a protein chip kit, but the present disclosure is not limited thereto.

According to yet another aspect of the present disclosure, there is provided a method of providing information for diagnosis of TKI-resistant cancer in a first patient with cancer (first cancer patient), including the following steps:

(a) measuring an expression level of mRNA of an ERRγ gene or a protein expressed therefrom in a biological sample isolated from the first cancer patient to check whether cancer exhibits resistance to TKI;

(b) measuring an expression level of mRNA of an ERRγ gene or a protein expressed therefrom in a biological sample isolated from a second patient with a cancer that is not resistant to TKI or a subject without a cancer; and

(c) determining the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

According to yet another aspect of the present disclosure, there is provided a method of providing information required to determine a therapeutic method for a first patient with a cancer (first cancer patient), including the following steps:

(a) measuring an expression level of mRNA of an ERRγ gene or a protein expressed therefrom in a biological sample isolated from the first cancer patient;

(b) measuring an expression level of mRNA of an ERRγ gene or a protein expressed therefrom in a biological sample isolated from a second patient with a cancer that is not resistant to TKI or a subject without a cancer; and

(c) judging the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

According to yet another aspect of the present disclosure, there is provided a method of diagnosing and treating TKI-resistant cancer in a first patient, including the following steps:

(a) measuring an expression level of mRNA of an ERRγ gene or a protein expressed therefrom in a biological sample isolated from the first patient;

(b) measuring an expression level of mRNA of an ERRγ gene or a protein expressed therefrom in a biological sample isolated from a second patient with a cancer that is not resistant to TKI or a subject without a cancer; and

(c) determining the first cancer patient in step (a) to be a patient with TKI-resistant cancer and applying another cancer therapeutic agent other than an TKI when the expression level of the mRNA of the ERRγ gene or the protein expressed therefrom measured in step (a) is higher than the expression level of the mRNA of the ERRγ gene or the protein expressed therefrom measured in step (b).

According to one embodiment of the present disclosure, the biological sample may include cancer tissue, cancer cells, whole blood, plasma, serum, or blood, but the present disclosure is not limited thereto.

According to yet another aspect of the present disclosure, the present disclosure also provides a method of screening a material that enhances the treatment of TKI-resistant cancer, which includes (a) treating TKI-resistant cancer cells with a candidate material; and (b) evaluating the activity or expression of ERRγ in the cells. A material capable of inhibiting the activity or expression of ERRγ in TKI-resistant cancer cells may be selected through such a method, and may be used as an enhancer or adjuvant for treatment of TKI-resistant cancer.

According to yet another aspect of the present disclosure, the present disclosure provides a kit for diagnosing TKI-resistant cancer.

Specifically, the kit for diagnosing TKI-resistant cancer includes an agent for measuring a level of mRNA of an ERRγ gene or a protein expressed therefrom.

In the present disclosure, the term “diagnosis” means confirming the presence or characteristics of a pathological condition. In the present disclosure, the diagnosis is to determine the presence or occurrence of TKI-resistant cancer by measuring a level of mRNA of an ERRγ gene or a protein expressed therefrom.

As used in the present disclosure, the term “an agent for measuring an expression level of mRNA of an ERRγ gene or a protein thereof” refers to a molecule that may be used to check an expression level of an ERRγ gene or a protein encoded by such a gene. In this case, the agent may preferably be a pair of primers, a probe, or an antisense nucleotide, all of which specifically bind to the ERRγ gene, or may be an antibody or an aptamer specific for a protein encoded by the ERRγ gene.

In the present disclosure, the term “primer” refers to a short nucleic acid sequence having a free 3′ hydroxyl group, that is, a short nucleic acid sequence that may form a base pair with a complementary template and serves as a starting point for copying the template. In the present disclosure, PCR amplification may be performed using sense and antisense primers of a marker polynucleotide according to the present disclosure to screen patients with TKI-resistant cancer through the production level of the desired product. The PCR conditions, the length of the sense and antisense primers can be modified based on what is known in the art.

As used in the present disclosure, the term “probe” refers to a nucleic acid fragment such as RNA or DNA corresponding to several bases to several hundred bases that may specifically bind to mRNA, and may be labeled to determine the presence/absence of specific mRNA. The probe may be manufactured in the form of an oligonucleotide probe, a single-stranded DNA probe, a double-stranded DNA probe, an RNA probe, and the like. In the present disclosure, hybridization may be performed using a probe complementary to the ERRγ polynucleotide of the present disclosure in order to screen patients with RAF inhibitor-resistant cancer based on the degree of hybridization. The selection of suitable probes and the hybridization conditions can be modified based on what is known in the art.

The primers or probes of the present disclosure may be chemically synthesized using a phosphoramidite solid support method or other well-known methods. Such nucleic acid sequences may also be modified using a number of means known in the art. Non-limiting examples of such modifications include methylation, encapsulation, substitution of one or more homologs of a natural nucleotide, and intemucleotide modifications such as modifications into uncharged linkages (e.g., methyl phosphonate, phosphotriester, phosphoramidate, carbamate, etc.) or charged linkages (e.g., phosphorothioate, phosphorodithioate, etc.).

A nucleotide sequence of the agent for measuring an expression level of the ERRγ gene used in the present disclosure is interpreted to include a sequence that exhibits substantial identity with a sequence specifically binding to the ERRγ gene in consideration of the mutations having biologically equivalent activity. The term “substantial identity” refers to a sequence that exhibits an identity of at least 60%, more specifically an identity of 70% or more, even more specifically an identity of 80% or more, and most specifically an identity of 90% or more, 91% or more, 92% or more, 93% or more, 94% or more, 95% or more, 96% or more, 97% or more, 98% or more, or 99% or more, when a specific sequence and any other sequence are aligned to the maximum possible correspondence and the aligned sequence is analyzed using an algorithm commonly used in the art.

In the kit of the present disclosure, the terms “antibody” and “aptamer” are the same as described in the pharmaceutical composition, and thus a description thereof will be omitted.

The kit of the present disclosure may be used to diagnose TKI-resistant cancer by checking an mRNA expression level of the ERRγ gene or an expression level of a protein encoded by the gene, or used to predict the therapeutic response to a TKI treatment.

Because the kit of the present disclosure may be used to predict the therapeutic response to TKI treatment, the kit may also be used for predicting the prognosis of cancer patients.

In the present disclosure, the term “predicting the prognosis of” refers to a process of guessing about a medical consequence in advance, and for the purpose of the present, means presuming the resistance of cancer patients to TKI treatment.

According to one embodiment of the present disclosure, the kit may be an RT-PCR kit, a competitive RT-PCR kit, a real-time RT-PCR kit, a DNA chip kit, or a protein chip kit, but the present disclosure is not limited thereto.

According to one embodiment of the present disclosure, the kit for measuring an expression level of mRNA of the ERRγ gene may be a kit including essential elements necessary for performing RT-PCR. In addition to each pair of primers specific for a marker gene, the RT-PCR kit contains test tubes or other suitable containers, a reaction buffer, deoxynucleotides (dNTPs), a TAQ™-polymerase and a reverse transcriptase, DNase, an RNase inhibitor, DEPC-water, sterile water, and the like.

The kit of the present disclosure may include a kit for extracting a nucleic acid (e.g., total RNA) from a body fluid, cells, or tissue, a fluorescent substance for labeling, an enzyme and medium for nucleic acid amplification, instructions for use, and the like.

According to another embodiment of the present disclosure, the kit of the present disclosure may be a kit for detecting ERRγ including essential elements necessary for using a DNA chip. The DNA chip kit may include a substrate to which cDNA corresponding to a gene or a fragment thereof is attached as a probe, and the substrate may include cDNA corresponding to a quantitative control gene or a fragment thereof.

According to still another embodiment of the present disclosure, the kit for measuring an expression level of the protein encoded by ERRγ according to the present disclosure may include a substrate for immunological detection of an antibody, a suitable buffer solution, a secondary antibody labeled with a chromogenic enzyme or a fluorescent substance, a chromogenic substrate, and the like. In this case, a nitrocellulose membrane, a 96-well plate synthesized from polyvinyl resin, a 96-well plate synthesized from polystyrene resin, and a glassy slide glass may be used as the substrate. A peroxidase and an alkaline phosphatase may be used as the chromogenic enzyme. FITC, RITC, and the like may be used as the fluorescent material, and ABTS (2,2′-azino-bis-(3-ethylbenzothiazoline-6-sulfonic acid)), OPD (o-phenylenediamine), TKIB (tetramethyl benzidine), and the like may be used as the chromogenic substrate.

The kit of the present disclosure may be configured to further include a composition, a solution or a device having one or more other components suitable for an analysis method.

According to still another aspect, the present disclosure provides a method of providing information for diagnosis of TKI-resistance cancer.

Specifically, the method of providing information for diagnosis of TKI-resistant cancer includes: (a) measuring an expression level of mRNA of an ERRγ gene or a protein expressed therefrom in a biological sample isolated from a first cancer patient to check whether cancer exhibits resistance to TKI; (b) measuring an expression level of mRNA of an ERRγ gene or a protein expressed therefrom in a biological sample isolated from a second patient with cancer that is not resistant to RAF inhibitor treatment or a subject without a cancer; and (c) determining the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

In the present disclosure, the term “biological sample” refers to a tissue (cancer tissue), cells (cancer cells), whole blood, plasma, serum, blood, saliva, synovial fluid, urine, sputum, lymph fluid, cerebrospinal fluid, tissue autopsy samples (brain, skin, lymph nodes, spinal cord, or the like), a cell culture supernatant, or ruptured eukaryotic cells, which have different expression and/or activity levels of ERRγ as a TKI-resistant cancer marker. Also, the biological sample includes samples derived from metastatic lesions as well as primary cancer lesions. In this case, the activity or expression level of ERRγ may be determined in a state in which these biological samples are manipulated or not manipulated.

In the method of providing information for diagnosis of RAF inhibitor-resistant cancer according to the present disclosure, the biological sample of step (a) may be obtained using a specific method known to those skilled in the art. For example, the biological sample may be obtained from a vertebrate, particularly a mammal, and may be preferably obtained from a cancer patient to check whether cancer exhibits resistance to RAF inhibitor.

In this case, the cancer patient is a human. A tissue biopsy is often used to obtain a representative piece of tumor tissue. Alternatively, the tumor cells may be indirectly obtained in the form of a tissue or a fluid known or believed to contain the tumor cells of interest.

In the present disclosure, the term “measuring an expression level of mRNA” refers to a process of confirming the presence and expression level of mRNA of the ERRγ gene in a biological sample, and may be known by measuring an amount of mRNA. Analysis methods for this include RT-PCR, competitive RT-PCR, real-time RT-PCR, an RNase protection method, northern blotting, or DNA chip technology, but the present disclosure is not limited thereto.

In the present disclosure, the term “measuring an expression level of a protein” refers to a process of confirming the presence and expression level of a protein expressed from the ERRγ gene in a biological sample. In this case, an amount of the protein may be determined using an antibody specifically binding to the protein expressed from the gene. Analysis methods for this include Western blotting, an enzyme linked immunosorbent assay (ELISA), a radioimmunoassay (RIA), radial immunodiffusion, Ouchterlony immunodiffusion, rocket immunoelectrophoresis, immunohistochemical staining, an immunoprecipitation assay, a complement fixation assay, immunofluorescence, immunochromatography, fluorescence-activated cell sorter analysis (FACS analysis), protein chip technology, or the like, but the present disclosure is not limited thereto.

In the method of providing information for diagnosis of TKI-resistant cancer according to the present disclosure, a patient with cancer that is not resistant to an TKI may be a patient with breast cancer, prostate cancer, melanoma, colorectal cancers, glioma (including recurrent glioblastoma), lung cancer, kidney cancer, ovarian cancer, sarcoma, desmoid tumor (aggressive fibromatosis), or thyroid cancer who does not exhibit TKI-resistant cancer. For example, in a TKI-resistant liver cancer, the patent with a cancer that is not resistant to an TKI may be a patient with hepatocellular carcinoma (HCC) or hepatic adenocarcinoma who does not exhibit TKI-resistant liver cancer, but the present disclosure is not limited thereto.

In the method of providing information for diagnosis of TKI-resistant cancer according to the present disclosure, the expression level of the ERRγ gene may be measured at an mRNA level or a protein level, and the isolation of the mRNA or the protein from the biological sample may be performed using a process known in the art. An analysis method of measuring an mRNA level and an analysis method of measuring a protein level are as described above.

Through the method of providing information for diagnosis of TKI-resistant cancer according to the present disclosure, it is possible to predict the therapeutic response of cancer patients to an TKI and establish a treatment strategy for each patient based on the prediction results, thereby making it possible to effectively treat cancer.

Therefore, according to yet another aspect, the present disclosure provides a method of providing information required to determine a therapeutic method for a cancer patient, which includes measuring an expression level of mRNA of an ERRγ gene or a protein expressed therefrom in a biological sample isolated from a cancer patient.

Specifically, the method of providing information required to determine a therapeutic method for a first cancer patient may include: (a) measuring an expression level of mRNA of an ERRγ gene or a protein expressed therefrom in a biological sample isolated from the first cancer patient; (b) measuring an expression level of mRNA of an ERRγ gene or a protein expressed therefrom in a biological sample isolated from a second patient with a cancer that is not resistant to TKI or a subject without a cancer; and (c) determining the first cancer patient in step (a) to be a patient with TKI-resistant cancer when the expression level of the mRNA of the ERRγ gene or the protein expressed therefrom measured in step (a) is higher than the expression level of the mRNA of the ERRγ gene or the protein expressed therefrom measured in step (b).

According to the method of providing information required to determine a therapeutic method for a cancer patient, when resistance to an TKI is confirmed in the cancer patient, a cancer treatment effect may be induced by applying other known cancer therapeutic agents other than TKI.

Therefore, according to a further aspect, the present disclosure provides a method of diagnosing and treating TKI-resistant cancer.

Specifically, the method for diagnosing and treating TKI-resistant cancer of a first patient according to the present disclosure includes: (a) measuring an expression level of mRNA of an ERRγ gene or a protein expressed therefrom in a biological sample isolated from a first cancer patient; (b) measuring an expression level of mRNA of an ERRγ gene or a protein expressed therefrom in a biological sample isolated from a second patient with cancer that is not resistant to TKI; and (c) determining the first cancer patient in step (a) to be a patient with TKI-resistant cancer and applying another cancer therapeutic agent other than TKI when the expression level of the mRNA of the ERRγ gene or the protein expressed therefrom measured in step (a) is higher than the expression level of the mRNA of the ERRγ gene or the protein expressed therefrom measured in step (b).

Specific details of the method for providing information required to determine a therapeutic method for a cancer patient and/or the method of diagnosing and treating TKI-resistant cancer according to the present disclosure are as described above in the method of providing information for diagnosis of TKI-resistant cancer, and a description thereof will be omitted.

It should be understood that the terms and words used in the specification and the appended claims should not be construed as being limited to general and dictionary meanings, but interpreted based on the meanings and concepts corresponding to technical aspects of the present disclosure on the basis of the principle that the present inventors can appropriately define the concepts of terms to describe the present disclosure in the best way.

Hereinafter, preferred embodiments are provided to aid in understanding the present disclosure. However, it should be understood that the following examples are merely intended to provide a better understanding of the present disclosure, and are not intended to limit the scope of the present disclosure.

### EXPERIMENTAL EXAMPLES

### General Experimental Methods

As an example of TKI-resistant cancer model, the present inventors employed sorafenib-resistant liver cancer cell lines (Huh7-SR and SK-Hep-R cell lines) and a liver cancer cell line-derived animal model (xenograft). To determine an effect of the synthetic compound DN200434 (compound of Formula 1a), which is an inverse agonist against ERRγ, on the proliferation of liver cancer cells and ROS generation, ROS was measured using a cell count and 2′,7-dichlorofluorescin diacetate (DCF-DA). Finally, after a sorafenib-resistant liver cancer cell line was injected into rats to induce the formation of sorafenib-resistant liver cancer, changes in size of liver cancer were confirmed in the group injected with the inverse agonist against ERRγ (DN434) and sorafenib and the group injected with the control drug.

### Example 1

### Construction of Sorafenib-Resistant Liver Cancer Cell Line and Confirmation of Increased ERRγ Expression

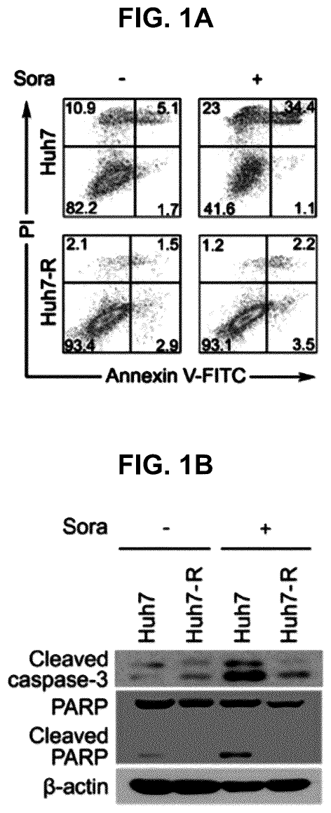

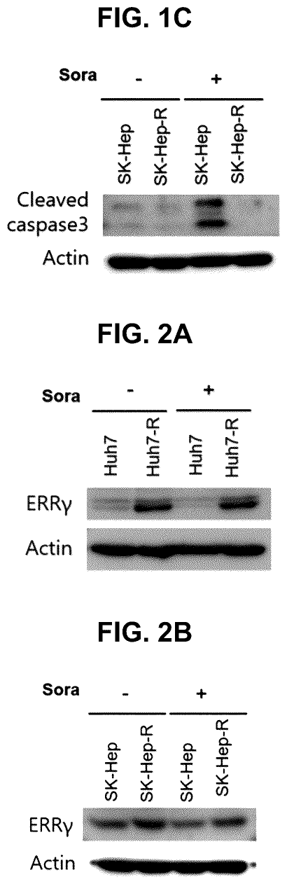

Hepatocarcinoma cell lines [Huh7 cells (Korea Cell Line Bank (KCLB) No. 60104), SK-Hep cells (ATCC° HTB-52™)] were continuously exposed to sorafenib (gradually increasing to 10 μM) to construct sorafenib-resistant liver cancer cell lines (Huh7-SR and SK-Hep-R cell lines). To evaluate the cancer cell death by FACS, first, a cancer cell line was incubated in FITC-bound annexin and propidium iodide (PI) for 15 minutes, and annexin and PI binding were then measured by flow cytometry to acquire data using a BD Accuri C6 flow cytometer (BD Biosciences) and analyzed with the ACCURI™ C6 analysis program (BD Biosciences)/FlowJo software (FlowJo, LLC.). Cancer cell line death was evaluated using a cleaved caspase-3 antibody (Cell Signaling Technology). As shown in FIGS. 1A to 1C, it was confirmed that the cell death by sorafenib did not increase in both the sorafenib-resistant liver cancer cell lines (e.g., Huh7-SR and SK-Hep-R cell lines).

Also, Western blotting was performed to confirm an expression pattern of the orphan nuclear receptor ERRγ in the Huh7-SR and SK-Hep-R cell lines. As a result, it was confirmed that the expression of ERRγ remarkably increased in both of the two sorafenib-resistant liver cancer cell lines, as shown in FIGS. 2A and 2B.

### Example 2

### Investigation of Effect of Orphan Nuclear Receptor ERRγ on Sorafenib-Resistant Liver Cancer

In order to investigate an effect of an ERRγ inhibitor, the compound (DN200434) represented by Formula 1a on ROS generation in sorafenib-resistant liver cancer cells, the effect was measured by FACS using 2′,7′-dichlorohydrofluorescein diacetate (H2-DCF-DA; Invitrogen, USA) as an ROS probe. Drug-resistant cells were treated with sorafenib (10 μM) and the DN200434 compound (12 μM) for 24 hours, and 10 μM H2-DCF-DA was added to the cells. Then, the cells were incubated for 30 minutes, and washed with PBS, and data was collected using a BD ACCURI™ C6 flow cytometer (BD Bioscience, USA), and analyzed using an ACCURI™ C6 analysis program (BD Bioscience, USA). To determine the number of cells, the sorafenib-resistant liver cancer cell line was treated with the DN200434 compound (12 μM) in combination with sorafenib (10 μM), and stained with trypan blue, and the number of cells was then measured using a hemocytometer.

As ERRγ inhibitors, the compounds of Formulas 1b-1d are tested in a same manner as described in Example 2.

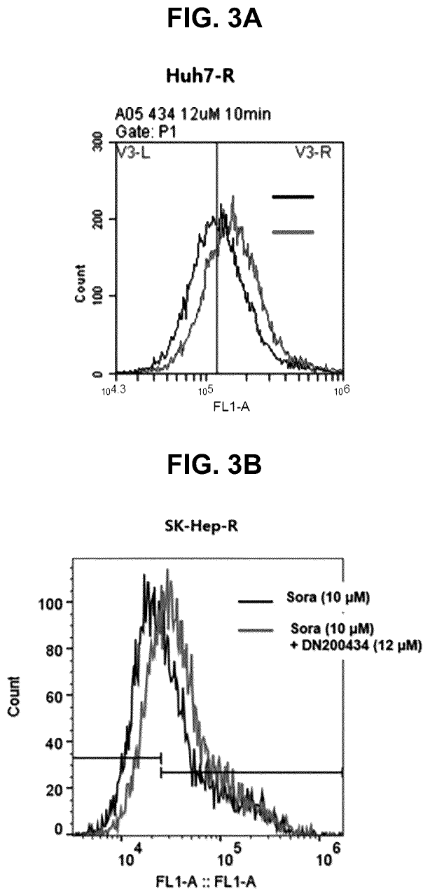

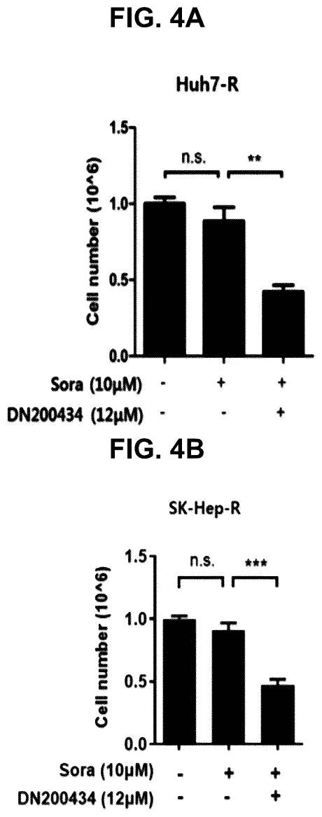

As a result for the compound of Formula 1a, as shown in FIGS. 3A and 3B, it was confirmed that an increase in ROS by the compound was observed in both the Huh?-SR and SK-Hep-R cell lines, which are sorafenib-resistant liver cancer cell lines. As shown in FIGS. 4A and 4B, it was confirmed that the cell proliferation of the sorafenib-resistant liver cancer cells, which was not affected when treated with sorafenib alone, significantly decreased by the simultaneous administration of the compound of Formula 1a. Based on the results as described above, it was confirmed that an ERRγ inhibitor might overcome drug resistance by inhibiting ERRγ activity and increasing the susceptibility to sorafenib.

### Example 3

### Confirmation of Anticancer Effect in Sorafenib-Resistant Liver Cancer Animal Model

The sorafenib-resistant liver cancer cell line Huh7-R was injected into mice in order to construct a sorafenib-resistant liver cancer animal model (xenograft). Thereafter, the inverse agonist against ERRγ (e.g., DN200434 (Formula 1a)) was administered in combination with sorafenib, and the size change of the formed mass was measured. In this case, the group in which the sorafenib-resistant liver cancer-derived animal model was treated with sorafenib alone was used as the comparative control.

As ERRγ inhibitors, the compounds of Formulas 1b-1d are tested in a same manner as described in Example 3.

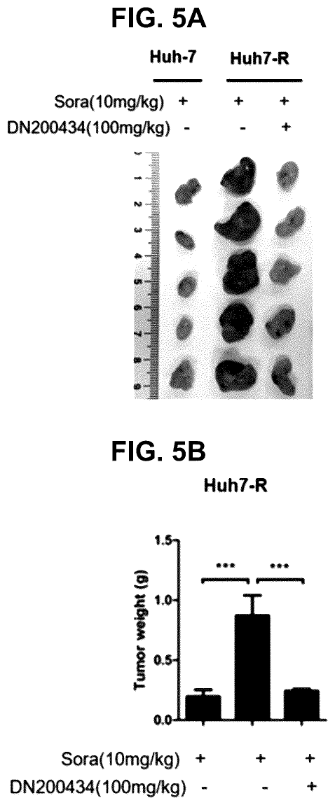

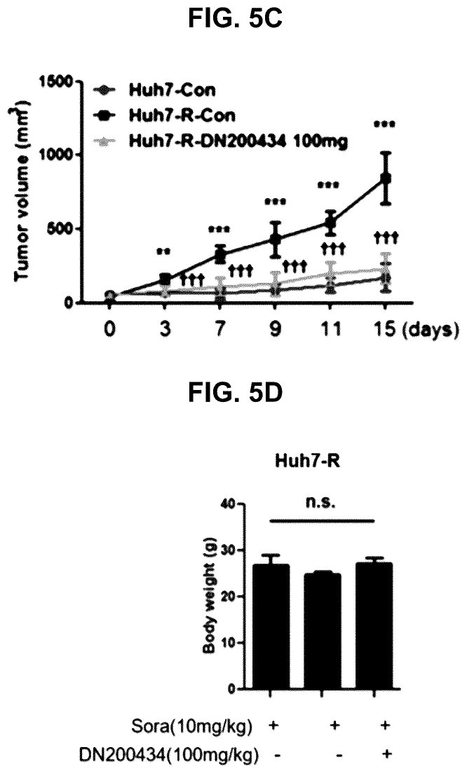

As a result of the compound of Formula la, as shown in FIGS. 5A to 5C, it was confirmed that the tumor size, weight and volume significantly decreased in the group in which the compound was administered in combination with sorafenib in the animal model derived from the sorafenib-resistant liver cancer cell line Huh?-R, compared to the control. In this case, as seen from FIG. 5D, it was confirmed that there was no difference in in body weight change between the experimental group and the control.

An animal model derived from the sorafenib-resistant liver cancer cell line SK-Hep-R was constructed in the same manner as described above, and the effect of co-administration of an ERRγ inhibitor and sorafenib was confirmed.

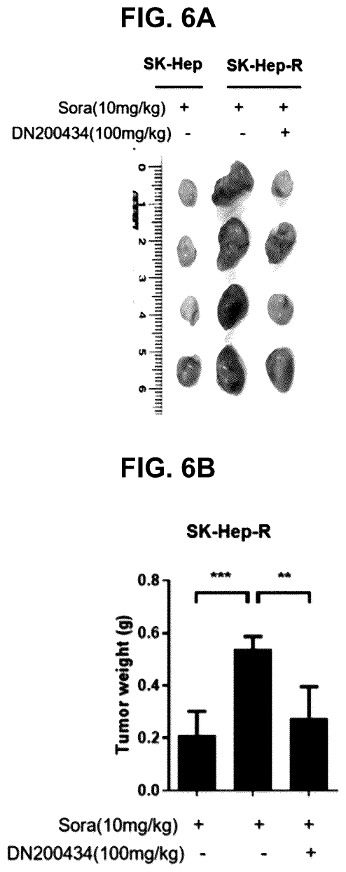

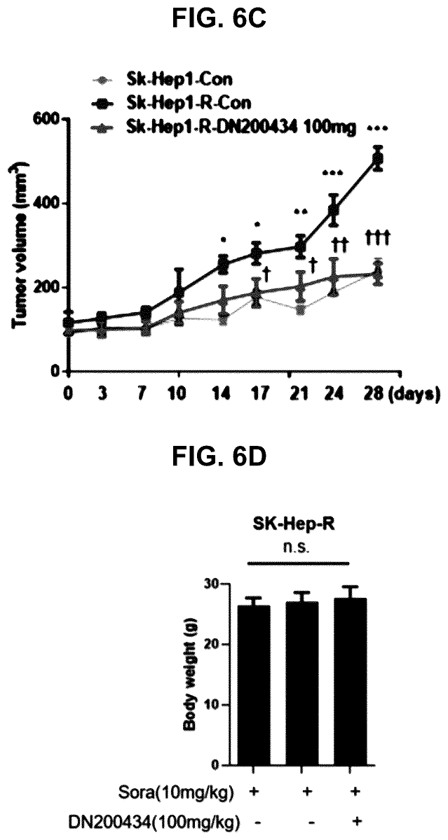

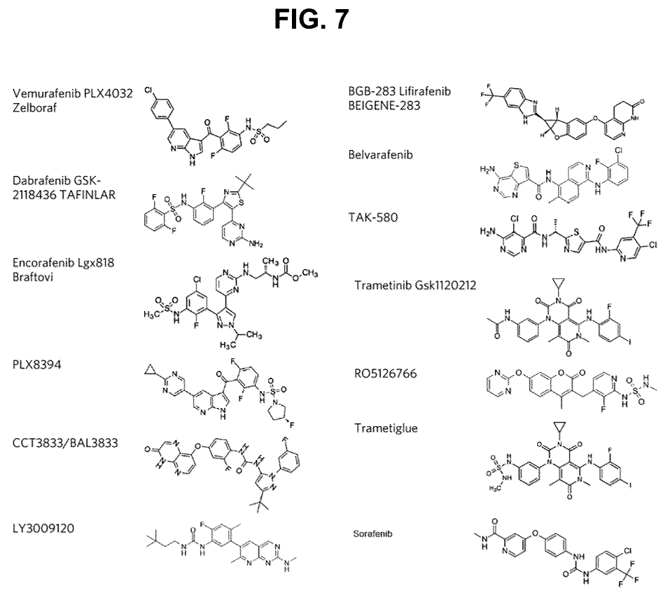

Similarly, as shown in FIGS. 6A to 6C, it was confirmed that the tumor size, weight, and volume of liver cancer remarkably decreased in the group in which an ERRγ inhibitor was administered in combination with sorafenib even in the animal model derived from the sorafenib-resistant liver cancer cell line SK-Hep-R, compared to the control. As seen from FIG. 6D, it was confirmed that there was no difference in in body weight change between the experimental group and the control.

Putting together the results of Examples 1 to 3, it was confirmed that the expression of ERRγ significantly increased in TKI-resistance cancer. Also, it was confirmed through the experiments that an ERRγ inhibitor increased intracellular ROS, inhibited the proliferation of the TKI-resistant cancer cells, and increased the susceptibility to TKI. In addition, it was also confirmed that the cancer proliferation significantly decreased, compared to the control, in an animal experiment using a TKI-resistant cancer cell line. The above results prove that ERRγ plays an important role in drug resistance in cancer, and is effective in treating advanced drug-resistant cancer when ERRγ activity is inhibited using an inverse agonist against ERRγ.

### Example 4

### Anticancer Effect in Erlotinib-Resistant Lung Cancer Cells

Erlotinib-resistant HCC827 cells were cultured in RPMI-1640 medium containing 10% FBS and 1% antibiotics.

To initiate the assay, cells were seeded in 96-well plates at a density of 1×10⁴ cells per well and incubated overnight in a 37° C. CO₂ incubator, followed by treating cells with DN434 for 24 hours. After 24 hours of incubation, the media was replaced with media containing 0.5 mg/ml of MTT and the plates were incubated for 1 hour. Then, the medium was removed and formazan crystals were dissolved in 100 μl of DMSO added to each well. A microplate reader was used to measure the absorbance at 570 nm, and cell viability was normalized to control. Statistical significance was calculated using Student's t-test, with significance levels set at **p<0.01, ***p<0.001, and ****p<0.0001. The half maximal inhibitory concentration (IC₅₀) was calculated.

The results are shown in FIG. 13. FIG. 13 demonstrates that the ERRγ compound (DN434) inhibited the growth of erlotinib-resistant HCC827 cells, with an IC₅₀ of 16.66 μM, indicating that DN434 is also effective in inhibiting the growth of erlotinib-resistant lung cancer cells.

### Example 5

### Synergistic Anticancer Effects by Combinations of ERRγ Compound and TKIs

To evaluate anticancer effects of ERRγ compound, alone and in combination with TKIs, various cancer cells including A549, Panc-1, Sk-Hep-1, Huh-7, and SK-Hep-1 cells were cultured in DMEM containing 10% FBS and 1% antibiotics. And, HCC827, PC9, and SKOV-3 cells were cultured in RPMI-1640 medium containing 10% FBS and 1% antibiotics.

To initiate the assay, cells were seeded in 96-well plates at a density of 1×10⁴ cells per well and incubated overnight in a 37° C. CO₂ incubator, followed by treating the cells with either TKIs, DN434, or a combination of both for 24 hours. To serve as a control, an equal volume of DMSO was added. After 24 hours of incubation, the media was replaced with media containing 0.5 mg/ml of MTT and the plates were incubated for 1 hour. Then, the medium was removed and formazan crystals were dissolved in 100 μl of DMSO added to each well. A microplate reader was used to measure the absorbance at 570 nm, and cell viability was normalized to control. Statistical significance was calculated using Student's t-test, with significance levels set at **p<0.01, ***p<0.001, and ****p<0.0001.

The results are shown in FIGS. 8-12. Based on the results presented in FIG. 8, the growth of the A549 lung cancer cell line was inhibited by 16.3%, 26.4%, and 83.7% by bosutinib (12.5 μM), DN434 (25 μM), and the combination of both, respectively. The results indicate a synergistic effect. The combination treatment was found to have a significant synergistic cancer cell growth inhibition effect.

As shown in FIG. 9, it was observed that the growth of the HCC827 lung cancer cell line was inhibited by 16.6%, 3.4%, and 32.2% by gefitinib (25 nM), DN434 (25 μM), and the combination of both, respectively, with a significant synergistic effect by the combination (left graph). And, the growth of PC9 lung cancer cell line was inhibited by 25.1%, 19%, and 47.8% by gefitinib (12.5 nM), DN434 (8 μM), and the combination of both, respectively, also with a significant synergistic effect by the combination (right graph).

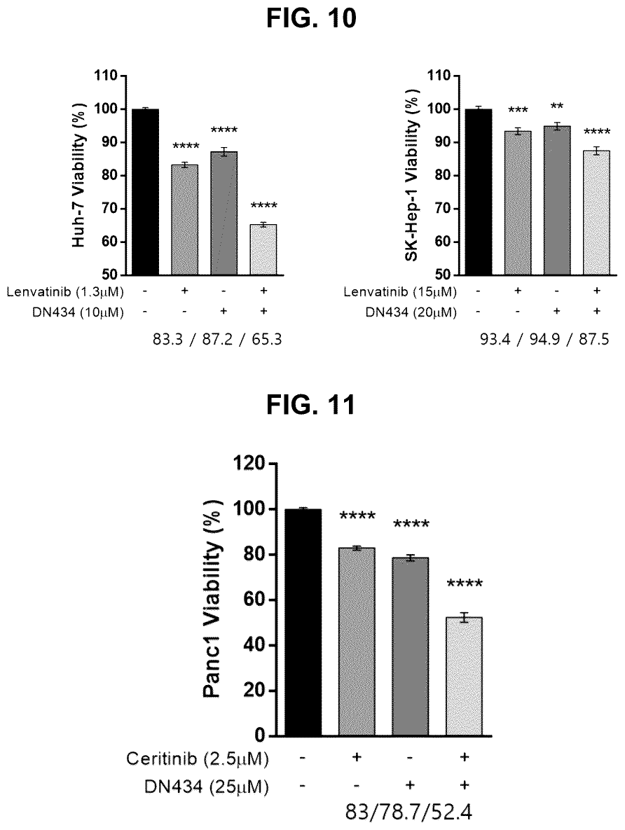

As demonstrated in FIG. 10, lenvatinib (1.3 μM), DN434 (10 μM), and the combination of both inhibited the growth of Huh-7 liver cancer cells by 16.7%, 12.8%, and 34.7%, respectively, with a significant synergistic effect by the combination (left graph). Similarly, in FIG. 3b, lenvatinib (15 μM), DN434 (20 μM), and the combination of both inhibited the growth of SK-Hep-1 liver cancer cells by 16.7%, 12.8%, and 34.7%, respectively, with a significant synergistic effect by the combination (right graph).

And, the results of FIG. 11 show that ceritinib (2.5 μM), DN434 (25 μM), and the combination of both inhibited the growth of Panc1 pancreatic cancer cells by 17.0%, 21.3%, and 47.6%, respectively, with a significant synergistic effect by the combination.

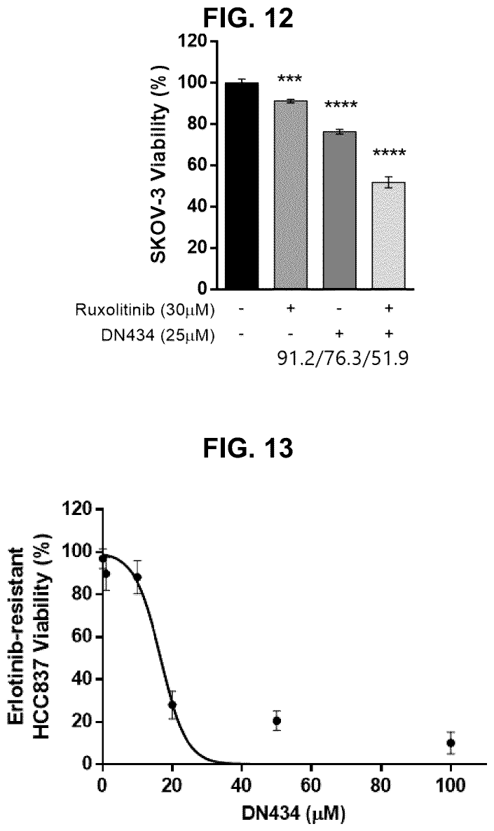

Additionally, the results of FIG. 12 show that ruxolitinib (30 μM), DN434 (25 μM), and the combination of both inhibited the growth of SKOV-3 ovarian cancer cells by 8.8%, 23.7%, and 48.1%, respectively, with a remarkable synergistic effect by the combination.

The above description of the present disclosure is given by way of illustration only, and it should be understood by those skilled in the art to which the present disclosure belongs that various changes and modifications can be made without departing from the technical spirit and scope of the present disclosure. Therefore, it should be understood that the aforementioned embodiments are given by way of illustration only, and are not intended to be limiting in all aspects.

1. A combination comprising (a) a first composition comprising an agent capable of inhibiting estrogen-related receptor γ (ERRγ) protein activity or ERRγ gene expression, and (b) a second composition comprising a tyrosine kinase inhibitor.

2. The combination of claim 1, wherein the agent for inhibiting ERRγ protein activity is an inverse agonist or an antagonist against ERRγ, or an antibody or an aptamer capable of specifically binding to ERRγ.

3. The combination of claim 2, wherein the inverse agonist or an antagonist against ERRγ is a compound of following Formula A: 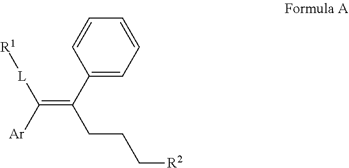
wherein
L is (C6-C20)arylene, (C3-C20)heteroarylene, or (C3-C20)fused heterocycle;
R¹ is (C3-C20)heterocycloalkyl, (C3-C20)heteroaryl, —O—(CH₂)ₘ—R¹¹, —(CH₂)ₘ—R¹², —NH—(CH₂)ₘ—R¹³, —NHCO—(CH₂)ₙ—R¹⁴, or —SiR¹⁶R¹⁷—(CH₂)ₘ—R¹⁵;
R¹¹ to R¹⁵ are independently of one another (C3-C20)heterocycloalkyl;
R¹⁶ and R¹⁷ are independently of each other (C1-C20)alkyl;
m is an integer of 1 to 3; and
n is an integer of 0 or 1;
Ar is (C6-C20)aryl or (C3-C20)heteroaryl, in which the aryl or heteroaryl of Ar may be further substituted by one or more selected from the group consisting of hydroxy, halogen, (C1-C20)alkyl, halo(C1-C20)alkyl, (C1-C20)alkoxy, nitro, cyano, —NR²¹R²², (C1-C20)alkylcarbonyloxy, (C1-C20)alkylcarbonylamino, guanidino, —SO₂—R²³, and —OSO₂—R²⁴;
R²¹ and R²² are independently of each other hydrogen, (C1-C20)alkylsulfonyl, or (C3-C20)cycloalkylsulfonyl;
R²³ and R²⁴ are independently of each other (C1-C20)alkyl, halo(C1-C20)alkyl, or (C3-C20)cycloalkyl;
R² is hydroxy, halogen, (C1-C20)alkylcarbonyloxy, or (C1-C20)alkylsulfonyloxy;
the heterocycloalkyl or heteroaryl of R¹ and the heterocycloalkyl of R¹¹ to R¹⁵ may be further substituted by one or more selected from the group consisting of (C1-C20)alkyl, (C3-C20)cycloalkyl, (C2-C20)alkenyl, amidino, (C1-C20)alkoxycarbonyl, hydroxy, hydroxy(C1-C20)alkyl, and di(C1-C20)alkylamino(C1-C20)alkyl; and
the heterocycloalkyl and heteroaryl contains one or more heteroatoms selected from the group consisting of N, O and S, and the heterocycloalkyl is a saturated or unsaturated mono-, bi-, or spirocycle having a carbon atom or nitrogen atom in a ring as a binding site,
an isomer, or a pharmaceutically acceptable salt thereof, or a solvate including a hydrate.

4. The combination of claim 3, wherein the compound of Formula A is a compound of the following Formulas 1a-1d, a pharmaceutically acceptable salt thereof, or an isomer thereof, or a solvate thereof: 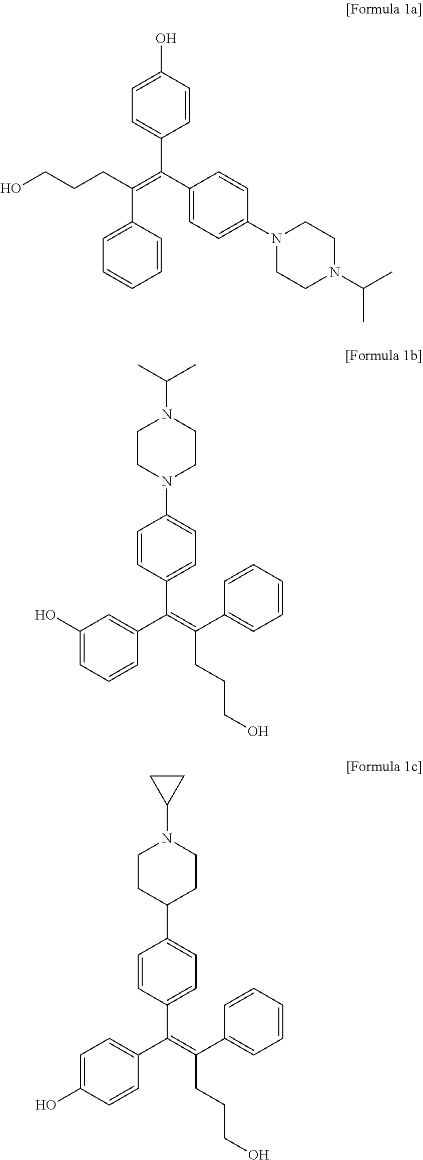 [SMILES: CC(C)N1CCN(c2ccc(/C(=C(/CCCO)c3ccccc3)c3ccc(O)cc3)cc2)CC1 ; CC(C)N1CCN(c2ccc(/C(=C(/CCCO)c3ccccc3)c3cccc(O)c3)cc2)CC1 ; OCCC/C(=C(\c1ccc(O)cc1)c1ccc(C2CCN(C3CC3)CC2)cc1)c1ccccc1] 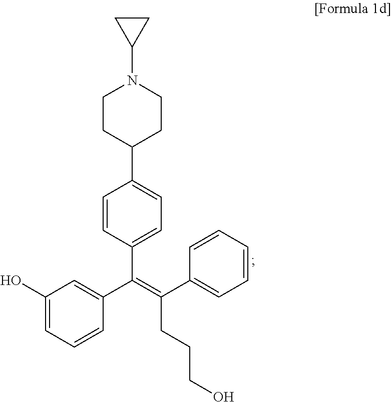 [SMILES: OCCC/C(=C(/c1ccc(C2CCN(C3CC3)CC2)cc1)c1cccc(O)c1)c1ccccc1]
or wherein the agent for inhibiting ERRγ gene expression is selected from the group consisting of miRNA, siRNA, shRNA, and an antisense oligonucleotide, all of which specifically bind to mRNA of the gene.

5. The combination of claim 1, wherein the tyrosine kinase inhibitor is bosutinib, gefitinib, lenvatinib, ceritinib, ruxolitinib, erlotinib, sunitinib, pazopanib, vandetanib, axitinib, cediranib, vatalanib, motesanib, sorafenib, afatinib, osimertinib, lazertinib, dabrafenib, encorafenib, lifirafenib, belvarafenib, trametinib, trametiglue, nazartinib, mavelertinib, azertinib, naquotinib, olmutinib, rociletinib, or a combination thereof.

6. The combination of claim 1, wherein the first composition is administered simultaneously with the second composition, separately, or sequentially.

7. A method of preventing or inhibiting tyrosine kinase resistance of a subject with cancer or enhancing a treatment of tyrosine kinase inhibitor-resistant cancer in a patient, comprising:
administering an effective amount of an agent capable of inhibiting estrogen-related receptor γ (ERRγ) protein activity or ERRγ gene expression to the subject.

8. The method of claim 7, wherein the agent for inhibiting ERRγ protein activity is an inverse agonist or an antagonist against ERRγ, or an antibody or an aptamer capable of specifically binding to the ERRγ.

9. The method of claim 8, wherein the inverse agonist or an antagonist against ERRγ is a compound of following Formula A: 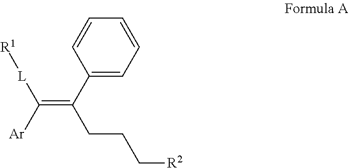
wherein
L is (C6-C20)arylene, (C3-C20)heteroarylene, or (C3-C20)fused heterocycle;
R¹ is (C3-C20)heterocycloalkyl, (C3-C20)heteroaryl, —O—(CH₂)ₘ—R¹¹, —(CH₂)ₘ—R¹², —NH—(CH₂)ₘ—R¹³, —NHCO—(CH₂)ₙ—R¹⁴, or —SiR¹⁶R¹⁷—(CH₂)ₘ—R¹⁵;
R¹¹ to R¹⁵ are independently of one another (C3-C20)heterocycloalkyl;
R¹⁶ and R¹⁷ are independently of each other (C1-C20)alkyl;
m is an integer of 1 to 3; and
n is an integer of 0 or 1;
Ar is (C6-C20)aryl or (C3-C20)heteroaryl, in which the aryl or heteroaryl of Ar may be further substituted by one or more selected from the group consisting of hydroxy, halogen, (C1-C20)alkyl, halo(C1-C20)alkyl, (C1-C20)alkoxy, nitro, cyano, —NR²¹R²², (C1-C20)alkylcarbonyloxy, (C1-C20)alkylcarbonylamino, guanidino, —SO₂—R²³, and —OSO₂—R²⁴;
R²¹ and R²² are independently of each other hydrogen, (C1-C20)alkylsulfonyl, or (C3-C20)cycloalkylsulfonyl;
R²³ and R²⁴ are independently of each other (C1-C20)alkyl, halo(C1-C20)alkyl, or (C3-C20)cycloalkyl;
R² is hydroxy, halogen, (C1-C20)alkylcarbonyloxy, or (C1-C20)alkylsulfonyloxy;
the heterocycloalkyl or heteroaryl of R¹ and the heterocycloalkyl of R¹¹ to R¹⁵ may be further substituted by one or more selected from the group consisting of (C1-C20)alkyl, (C3-C20)cycloalkyl, (C2-C20)alkenyl, amidino, (C1-C20)alkoxycarbonyl, hydroxy, hydroxy(C1-C20)alkyl, and di(C1-C20)alkylamino(C1-C20)alkyl; and
the heterocycloalkyl and heteroaryl contains one or more heteroatoms selected from the group consisting of N, O and S, and the heterocycloalkyl is a saturated or unsaturated mono-, bi-, or spirocycle having a carbon atom or nitrogen atom in a ring as a binding site,
an isomer, or a pharmaceutically acceptable salt thereof, or a solvate including a hydrate.

10. The method of claim 9, wherein the inverse agonist against ERRγ is a compound of the following Formulas 1a-1d, a pharmaceutically acceptable salt thereof, or an isomer thereof, or a solvate thereof:

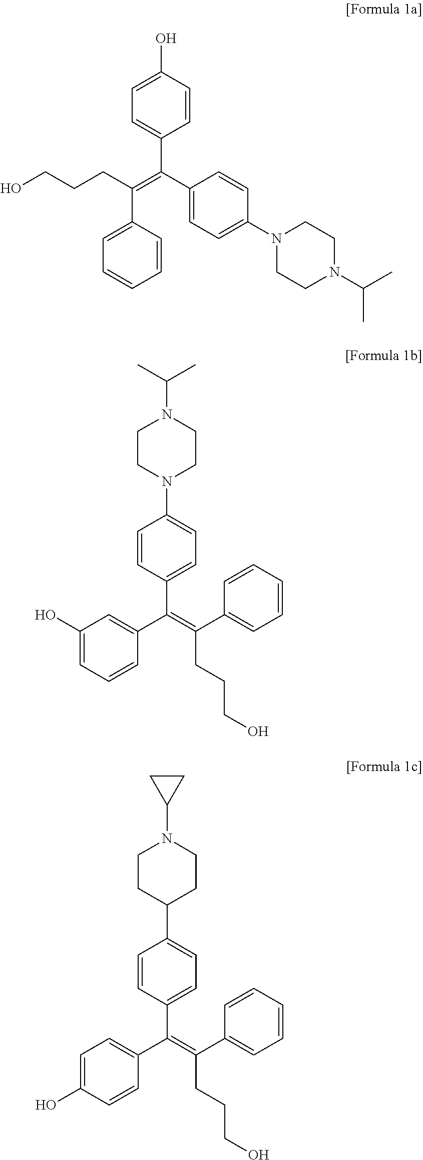 [SMILES: CC(C)N1CCN(c2ccc(/C(=C(/CCCO)c3ccccc3)c3ccc(O)cc3)cc2)CC1 ; CC(C)N1CCN(c2ccc(/C(=C(/CCCO)c3ccccc3)c3cccc(O)c3)cc2)CC1 ; OCCC/C(=C(\c1ccc(O)cc1)c1ccc(C2CCN(C3CC3)CC2)cc1)c1ccccc1]

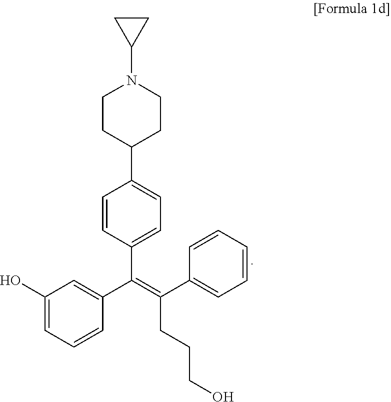 [SMILES: OCCC/C(=C(/c1ccc(C2CCN(C3CC3)CC2)cc1)c1cccc(O)c1)c1ccccc1]

11. The method of claim 7, wherein the agent for inhibiting ERRγ gene expression is selected from the group consisting of miRNA, siRNA, shRNA, and an antisense oligonucleotide, all of which specifically bind to mRNA of the gene.

12. The method of claim 7, which further comprises administering a tyrosine kinase inhibitor, simultaneously, separately, or sequentially.

13. A method of treating cancer in a subject in need thereof, comprising:
administering to the subject a first composition comprising an agent capable of inhibiting estrogen-related receptor γ (ERRγ) protein activity or ERRγ gene expression; and a second composition comprising a tyrosine kinase inhibitor.

14. The method of claim 13, wherein the subject has undergone tyrosine kinase inhibitor treatment.

15. The method of claim 13, wherein the subject has not undergone tyrosine kinase inhibitor treatment.

16. The method of claim 13, wherein the agent for inhibiting ERRγ protein activity is an inverse agonist or an antagonist against ERRγ, or an antibody or an aptamer capable of specifically binding to the ERRγ.

17. The method of claim 16, wherein the inverse agonist or an antagonist against ERRγ is a compound of following Formula A: 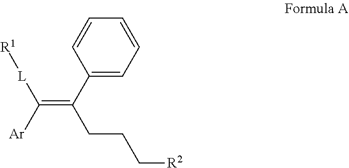
wherein
L is (C6-C20)arylene, (C3-C20)heteroarylene, or (C3-C20)fused heterocycle;
R¹ is (C3-C20)heterocycloalkyl, (C3-C20)heteroaryl, —O—(CH₂)ₘ—R¹¹, —(CH₂)ₘ—R¹², —NH—(CH₂)ₘ—R¹³, —NHCO—(CH₂)ₙ—R¹⁴, or —SiR¹⁶R¹⁷—(CH₂)ₘ—R¹⁵;
R¹¹ to R¹⁵ are independently of one another (C3-C20)heterocycloalkyl;
R¹⁶ and R¹⁷ are independently of each other (C1-C20)alkyl;
m is an integer of 1 to 3; and
n is an integer of 0 or 1;
Ar is (C6-C20)aryl or (C3-C20)heteroaryl, in which the aryl or heteroaryl of Ar may be further substituted by one or more selected from the group consisting of hydroxy, halogen, (C1-C20)alkyl, halo (C1-C20)alkyl, (C1-C20)alkoxy, nitro, cyano, —NR²¹—R²², (C1-C20)alkylcarbonyloxy, (C1-C20)alkylcarbonylamino, guanidino, —SO₂—R²³, and —OSO₂—R²⁴;
R²¹ and R²² are independently of each other hydrogen, (C1-C20)alkylsulfonyl, or (C3-C20)cycloalkylsulfonyl;
R²³ and R²⁴ are independently of each other (C1-C20)alkyl, halo(C1-C20)alkyl, or (C3-C20)cycloalkyl;
R² is hydroxy, halogen, (C1-C20)alkylcarbonyloxy, or (C1-C20)alkylsulfonyloxy;
the heterocycloalkyl or heteroaryl of R¹ and the heterocycloalkyl of R¹¹ to R¹⁵ may be further substituted by one or more selected from the group consisting of (C1-C20)alkyl, (C3-C20)cycloalkyl, (C2-C20)alkenyl, amidino, (C1-C20)alkoxycarbonyl, hydroxy, hydroxy(C1-C20)alkyl, and di(C1-C20)alkylamino(C1-C20)alkyl; and
the heterocycloalkyl and heteroaryl contains one or more heteroatoms selected from the group consisting of N, O and S, and the heterocycloalkyl is a saturated or unsaturated mono-, bi-, or spirocycle having a carbon atom or nitrogen atom in a ring as a binding site,
an isomer, or a pharmaceutically acceptable salt thereof, or a solvate including a hydrate.

18. The method of claim 17, wherein the inverse agonist against ERRγ is a compound of the following Formulas 1a-1d, a pharmaceutically acceptable salt thereof, or an isomer thereof, or a solvate thereof:

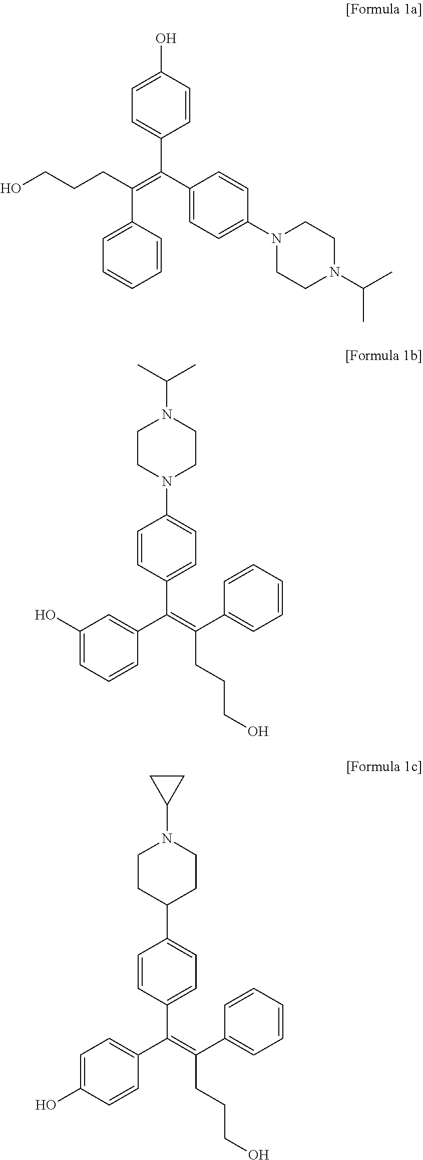 [SMILES: CC(C)N1CCN(c2ccc(/C(=C(/CCCO)c3ccccc3)c3ccc(O)cc3)cc2)CC1 ; CC(C)N1CCN(c2ccc(/C(=C(/CCCO)c3ccccc3)c3cccc(O)c3)cc2)CC1 ; OCCC/C(=C(\c1ccc(O)cc1)c1ccc(C2CCN(C3CC3)CC2)cc1)c1ccccc1]

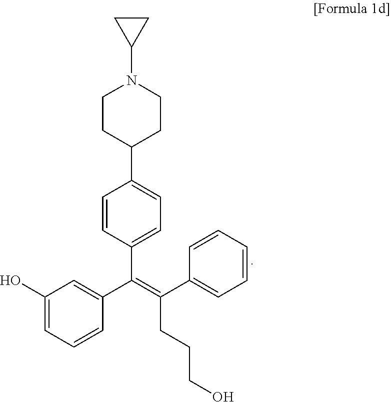 [SMILES: OCCC/C(=C(/c1ccc(C2CCN(C3CC3)CC2)cc1)c1cccc(O)c1)c1ccccc1]

19. The method of claim 13, wherein the agent for inhibiting ERRγ gene expression is selected from the group consisting of miRNA, siRNA, shRNA, and an antisense oligonucleotide, all of which specifically bind to mRNA of the gene.

20. The method of claim 13, wherein the first composition and the second composition are administered simultaneously, separately, or sequentially.

21. The method of claim 13, wherein the tyrosine kinase inhibitor is bosutinib, gefitinib, lenvatinib, ceritinib, ruxolitinib, erlotinib, sunitinib, pazopanib, vandetanib, axitinib, cediranib, vatalanib, motesanib, sorafenib, afatinib, osimertinib, lazertinib, dabrafenib, encorafenib, lifirafenib, belvarafenib, trametinib, trametiglue, nazartinib, mavelertinib, azertinib, naquotinib, olmutinib, rociletinib, or a combination thereof.

22. The method of claim 13, wherein the cancer is breast cancer, prostate cancer, melanoma, colorectal cancers, glioma (including recurrent glioblastoma), lung cancer, liver cancer, kidney cancer, ovarian cancer, sarcoma, pancreatic, desmoid tumor (aggressive fibromatosis), or thyroid cancer.

23. The method of claim 13, which further comprising administering a third composition and/or applying irradiation therapy, wherein the third composition comprises a therapeutic antibody.
